(19) United States
(12) Patent Application Publication
(10) Pub. No.: US 2022/0118159 A1
(43) Pub. Date: Apr. 21, 2022

(54) ANTIOXIDANT-RELEASING VITREOUS SUBSTITUTES AND USES THEREOF

(71) Applicant: Ohio State Innovation Foundation, Columbus, OH (US)

(72) Inventors: Katelyn Elizabeth REILLY, Upper Arlington, OH (US); Matthew Aaron REILLY, Upper Arlington, OH (US); Nguyen Khoi TRAM, Reynoldsburg, OH (US)

(21) Appl. No.: 17/428,617

(22) PCT Filed: Aug. 10, 2020
(86) PCT No.: PCT/US2020/017525

§ 371 Date: Aug. 4, 2021

Related U.S. Application Data
(60) Provisional application No. 62/944,679, filed on Dec. 6, 2019.
(60) Provisional application No. 62/926,267, filed on Oct. 25, 2019.
(60) Provisional application No. 62/803,419, filed on Feb. 8, 2019.

(51) Int. Cl. A61L 27/52; C08L 33/26; C08F 261/04; A61L 27/48; A61L 27/18

(52) U.S. Cl. CPC A61L 27/52; C08L 33/26; C08F 261/04; A61L 2300/428; A61L 27/18; A61L 2430/16; A61L 27/48

(57) ABSTRACT

In one aspect, the disclosure relates pertains to a vitreous substitute comprising a gel and an antioxidant, wherein the vitreous substitute mimics the physical properties of natural vitreous humor, as well as its methods of use in the treatment of ophthalmological disorders.

### CROSS-REFERENCE TO RELATED APPLICATIONS

This application claims the benefit of priority to U.S. Provisional Application No. 62/803,419, filed Feb. 8, 2019, U.S. Provisional Application No. 62/926,267, filed Oct. 25, 2019, and U.S. Provisional Application No. 62/944,679, filed Dec. 6, 2019, the disclosures of which are each incorporated herein by reference in their entirety.

### TECHNICAL FIELD

This disclosure related to vitreous substitutes, and more particularly to vitreous substitutes comprising a gel and an antioxidant.

### BACKGROUND

The vitreous humor is a fragile, transparent tissue between the lens and the retina, occupying 80% of the eye's volume. The vitreous serves as a mechanical cushion for the eye, absorbing impacts and protecting the lens and retina (Swindle-Reilly K E, et al. Biomaterials and regenerative medicine in ophthalmology. Woodhead Publishing. 2016). However, the vitreous degrades with age, which compromises its function as a shock absorber and causes complications such as retinal tear or detachment (Los L I, et al. Invest Ophthalmol Vis Sci. 2003; 44:2828-2833). Aside from its mechanical function, the natural vitreous also has other chemical functionalities, notably its role in oxygen homeostasis. Both the vitrectomy operation and replacement with substitutes including silicone oil disrupt this oxygen homeostasis, causing oxidative damage to intraocular tissues. In particular, oxidative damage to the lens results in cataract formation—up to 95% of patients require cataract extraction within 24 months after vitrectomy (Feng H, Adelman R A. Clin Ophthalmol. 2014; 8:1957-1965). Neither the current gold standard, silicone oil, nor other experimental vitreous substitutes address this problem.

Despite advances in research direct to vitreous substitutes for delivery of therapeutically useful compounds, there is still a scarcity of materials that are safe and efficacious. These needs and other needs are satisfied by the present disclosure.

### SUMMARY

In accordance with the purpose(s) of the disclosure, as embodied and broadly described herein, the disclosure, in one aspect, relates pertains to an ophthalmological composition comprising a disclosed vitreous substitute composition, wherein the vitreous substitute composition comprises a disclosed gel, hydrogel, or particle and a therapeutic agent, wherein the therapeutic agent is a disclosed antioxidant; methods of treating an ophthalmological disorder using a disclosed vitreous substitute; and methods of making a disclosed hydrogel comprising a polymer comprising residues of HEMA, PEGDA, and/or PEGMA.

Thus in one aspect, a vitreous substitute is provided comprising a gel and at least one antioxidant, wherein the vitreous substitute is defined by having a loss tangent (i.e., the ratio of loss modulus to storage modulus) of less than 1 (for example, a loss tangent ranging from 0.1 to 0.5) and a refractive index from about 1.33 to about 1.34.

In some embodiments, the vitreous substitute has a storage modulus ranging from 0.1 Pa to about 1000 Pa, for example from 1 Pa to about 100 Pa. In some embodiments, the vitreous substitute has a loss modulus ranging from about 0.01 Pa to about 1000 Pa, for example from about 0.1 Pa to about 100 Pa or from 0.1 Pa to about 50 Pa.

In some embodiments, the vitreous substitute has a refractive index from about 1.331 to about 1.339, for example from about 1.334 to about 1.337.

In some embodiments, the gel comprises a hydrogel. In some embodiments, the vitreous substitute comprises greater than 90% by weight water, for example greater than 95% by weight water.

In some embodiments, the hydrogel comprises a polymer composition. In some embodiments, the polymer composition may comprise one or more residues selected from a vinyl alcohol residue, an acrylate or methacrylate residue, an acrylamide residue, a residue derived from a functionalized polyethylene glycol, or combinations thereof. In some embodiments, the polymer composition may comprise one or more residues selected from acrylamide, N-ornithine acrylamide, N-(2-hydroxypropyl)acrylamide, hydroxyethylacrylate, hydroxyethylmethacrylate, polyethyleneglycol acrylates, polyethylene glycol methacrylates, N-vinylpyrrolidone, N-phenylacrylamide, dimethylaminopropyl methacrylamide, acrylic acid, benzylmethacrylamide, methylthioethylacrylamide, or combinations thereof.

In some embodiments, the polymer composition comprises one or more residues selected from poly(ethylene glycol)diacrylate (PEGDA), poly(ethylene glycol)methacrylate (PEGMA), 2-hydroxyethylmethacrylate (HEMA), or combinations thereof. In some embodiments, the polymer composition comprises a PEGMA:PEGDA copolymer. In some embodiments, the polymer composition comprises a PEGMA:PEGDA:HEMA copolymer.

In some embodiments, the hydrogel is loaded with the at least one antioxidant. In other embodiments, the vitreous substitute further comprises a particle, for example a nanoparticle. In some embodiments, the particle comprises chitosan, gelatin, alginate, or combinations thereof. In some embodiments, the particle encapsulates the at least one antioxidant.

In some embodiments, the at least one antioxidant can comprise: ascorbic acid or a derivative thereof; N-acetylcysteine; a glutathione; N-selenous acid; sodium selenite; L-carnitine; beta carotene; vitamin E; vitamin C; lutein; zeaxanthin; a zinc compound; a copper compound; an omega-3 fatty acid (such as DHA or EPA); alpha lipoid acid, or combinations thereof.

In some embodiments, the at least one antioxidant can comprise: alpha lipoic acid, ascorbic acid, riboflavin, glutathione, taurine, uric acid, tyrosine, transferrin, selenium, zinc, superoxide dismutase, glutathione peroxidase, catalase, pigment epithelium-derived factor (PEDF), derivatives thereof, or combinations thereof.

In some embodiments, the vitreous substitute of the present disclosure may further comprise one or more additional therapeutic agents as described herein. In some embodiments, the one or more additional therapeutic agents may comprise an anti-VEGF agent, a beta-adrenergic antagonist, a miotic, a carbonic anhydrase inhibitor, a prostaglandin, a serotonergic, a muscarinic, a dopaminergic agonist, an adrenergic agonist, an anti-angiogenesis agent, an anti-infective agent, a steroid, a non-steroidal anti-inflammatory drug, a growth factor, an immunosuppressant agent, an anti-allergic agent, or combinations thereof.

In another aspect, a method for treating an ophthalmological disorder in the eye of a subject in need thereof is provided, the method comprising injecting into the eye of the subject a therapeutically effective amount of the vitreous substitute as described herein. In some embodiments, the ophthalmological disorder may include macular degeneration, a retinal tear, or proliferative retinopathy. In some embodiments, the subject has been diagnosed with or is at risk of developing a cataract. In some embodiments, the vitreous substitute is administered following a vitrectomy.

Other systems, methods, features, and advantages of the present disclosure can be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a schematic representation for preparation and use of a disclosed PHEMA/PVA hydrogel vitreous substitute.

FIGS. 2A-2E shows representative images and data pertaining to a disclosed HEMA:PEGMA:PEGDA hydrogel vitreous substitute. FIG. 2A disclosed hydrogels loaded in syringes. FIG. 2B shows a representative image showing that a disclosed hydrogel retained its gel-like consistency after injection through a small-gauge needle. FIG. 2C shows rheological test apparatus with the hydrogel sandwiched between the parallel plate geometry and testing stage. A humidifying chamber (only shown in half) filled with phosphate buffered saline was used to prevent dehydration of the hydrogel sample during testing. FIG. 2D shows representative rheology data demonstrating viscoelasticity. FIG. 2E shows representative data for ascorbic acid release from disclosed gelatin-alginate particles demonstrating sustained release with concentration maintained around 2 mM for >30 days. HEMA: 2-Hydroxyethyl methacrylate; PEGDA: poly(ethylene glycol) diacrylate; and PEGMA: Poly(ethylene glycol) methacrylate.

FIGS. 3A-3B show, respectively, a schematic representations of a hydrogel vitreous substitute and vitreous humor with an oxygen gradient and effects of aging on the vitreous. FIG. 3A shows a schematic representation of shear thinning hydrogel vitreous substitute with nanoencapsulated ascorbic acid. FIG. 3B shows a schematic representation of vitreous humor composed of a network of collagen fibers and hyaluronic acid. The natural vitreous establishes an internal oxygen gradient with a high level of oxygen near the metabolically active retina and ciliary body and a low level of oxygen near the lens. However, the vitreous phase separates with age, disrupting its protective functions in the eye both physically and chemically. Some complications due to vitreous degradation include retinal detachment, retinal tear, and cataract formation.

FIG. 4 shows representative data for ascorbic acid release from a disclosed gelatin-alginate articles demonstrating burst release with concentration maintained around 2 mM.

FIG. 5 shows representative data for the release of ascorbic acid from a representative disclosed hydrogel. PEGMA hydrogel (20 ml, 5% v/v, MW 500) was synthesized then submerged in vitamin C solution (50 ml, 100 mM) for 12 h at room temperature. The hydrogel was placed in dialysis tubing and submerged in phosphate buffered saline (PBS, 70 ml). At predetermined times, the absorbance of PBS was measured at 265 nm to calculate the concentration of vitamin C release from PEGMA hydrogel. The data show that the concentration of vitamin C released spiked to 50 mM within the first day, then rapidly diminished to near zero on subsequent days.

FIG. 6 shows representative data for the release of vitamin C from vitamin C-loaded gelatin-alginate particles that were injected with a disclosed hydrogel through a 21G needle. The hydrogel/particles mixture were then submerged in PBS and the concentration of vitamin C in PBS was determined as aforementioned. The result showed a small spike in the release of vitamin C (compare to release from pure hydrogel above), followed by a period of sustained release of vitamin C as shown.

FIG. 7 shows representative data pertaining to the degradation of sodium ascorbate solutions. The data show a representative degradation profile of 2 mM sodium ascorbate solutions (n=3) and sodium ascorbate release profile from polyacrylamide hydrogels (n=3) at 37° C. with constant stirring. The polymer solutions with sodium ascorbate gelled within 18 hours. However, the polymer solutions without sodium ascorbate took twice as long to gel.

FIG. 8 shows representative data for release of sodium ascorbate from a disclosed polyacrylamide gel in terms of percent of sodium ascorbate released from polyacrylamide gel over 3 days, compared to the concentration of the 2 mM sodium ascorbate solutions at time 0 (which was 1.4 mM). Sodium ascorbate appeared to be fully released by the end of the first day. The percent drug release on the third day appeared to decrease due to the degradation of sodium ascorbate.

FIG. 9 shows representative data for release of sodium ascorbate from a disclosed chitosan particle composition. The study was done at room temperature with agitation (orbital shaker). The subsequent washing steps after the formation of chitosan particles likely diminished the actual amount of sodium ascorbate loaded in the particles. The data show a sustained released compared to the release profile from polyacrylamide hydrogels, with the sodium ascorbate continuing to be released even after 7 days.

FIGS. 10A-10D show representative rheological data for representative disclosed hydrogels (n=3). FIG. 10A shows representative data obtained in amplitude sweep experiments showed that the linear viscoelastic region of the hydrogels was below 10% strain. FIG. 10D shows representative data obtained in frequency sweep experiments showed that the hydrogels have similar storage modulus (G′) and loss modulus (G″) as the natural human vitreous. FIG. 10C shows representative data obtained in shear rate ramp experiments suggest that both hydrogels have shear-thinning behavior. FIG. 10D shows representative data obtained in alternating oscillatory step strain experiments further showed that both hydrogels could recover their gel-like behavior after undergoing large deformations. These results suggest that the hydrogels could be injected into the vitreal chamber using a syringe equipped with a small-gauge needle.

FIG. 11 shows transmittance data obtained for disclosed hydrogels. The hydrogels were as transparent as the natural human vitreous (n=3). The natural vitreous transmits 90% of light between 300 and 900 nm and none below this range. The hydrogels were at or above 90% transparency within the visible and infrared spectra. The transmittance of the hydrogels decreases in the ultra-violet range, dropping to zero at 230 nm.

FIG. 12 shows representative Fourier transform infrared (“FTIR”) spectroscopic data obtained for disclosed hydrogels. The FTIR data show successful synthesis of the PEGDA and PEGDA-co-PEGMA hydrogels. The methylene (—CH2-), carbonyl (C═O), and ether (C—O—C) groups were found in both hydrogel spectra at 2850, 1730, and 945 cm′, respectively. The alcohol (—OH) and methyl (—CH3) groups at 3740 and 1520 cm′, respectively, were only found in the PEGDA-co-PEGMA hydrogel spectra and not in the PEGDA spectra, confirming that the appropriate hydrogels were synthesized.

FIG. 13A-13B show representative stability data for representative disclosed hydrogels under different conditions as indicated. FIG. 13A shows stability data obtained for a disclosed PEGDA hydrogel. FIG. 13B shows stability data obtained for a disclosed PEGDA-co-PEGMA hydrogel. The data show that the water content of the hydrogels did not change for at least 28 days in DPBS, lysozyme, or trypsin solutions (n=3). This showed that the hydrogels were stable in enzymatic solutions and might be used as mid- to long-term vitreous substitutes.

FIG. 14A-14B show representative data for amount remaining and release of vitamin C from disclosed representative hydrogels as indicated versus time. FIG. 14A shows the amount of vitamin C remaining in disclosed representative hydrogels as indicated versus time. FIG. 14B shows the amount of vitamin C released from disclosed representative hydrogels as indicated versus time. The data show that vitamin C rapidly degraded or released from the hydrogels within the first 8 hours (n=3). The concentration approached zero after 7 days.

FIG. 15A-15B show representative in vitro cytotoxicity data for different cell types exposed to representative disclosed hydrogels as indicated. FIG. 15A shows representative in vitro cytotoxicity data for ARPE-19 cells exposed to representative disclosed hydrogels as indicated versus a media only control. FIG. 15B shows representative in vitro cytotoxicity data for LEC cells exposed to representative disclosed hydrogels as indicated versus a media only control. The data show that both hydrogels showed minimal in vitro cytotoxicity to ARPE-19 and LECs. Hydrogen peroxide treatment significantly decreased the cell viability of LECs compared to control. However, cell viability of ARPE-19 was equal to or greater with hydrogen peroxide treatment compared to control. Means that do not share a letter are significantly different (p<0.001, n=8). ARPE-19 cells are a human retinal pigmented epithelial cell line and are further described in the Examples. LEC cells are an immortalized human lens epithelial cell line and are further described in the Examples.

FIG. 16 shows representative data pertaining to the protective effect of disclosed hydrogels comprising vitamin C to reactive oxygen species (ROS). The presence of hydrogels and vitamin C had a synergistic effect on reducing ROS activity in ARPE-19 and LECs. Compared to control, hydrogen peroxide treatment did not increase ROS activity in ARPE-19, but statistically increased the ROS activity in LECs. Means that do not share a letter are significantly different (p<0.001, n=8).

FIG. 17A-17C shows representative images of injected porcine eyes. As shown, the PEGDA and PEGDA-co-PEGMA hydrogels could be injected into the vitreal chamber of porcine eyes and appeared to be similar to the natural vitreous. The porcine eyes used are as described in Examples.

FIG. 18 shows representative data for release of ascorbic acid from representative disclosed particles comprising ascorbic acid loaded chitosan particles coated with alginate, chitosan, and/or gelatin as indicated. The legend in the figure uses the following abbreviations for detailing the composition of the particle: VC denotes vitamin C; CH denotes chitosan; AL denotes alginate; GE denotes gelatin; and “GXXX” denotes glutathione, with the concentration (μM) indicated by the number “XXX” as shown. The particles were prepared as described in the examples.

FIG. 19 shows the data in FIG. 18, but with the vitamin C concentrations were normalized to the concentration at day 0.

FIG. 20 shows representative data for the stability of ascorbic acid from PEGDA and PEGDA-co-PEGMA hydrogels either without further additives, stabilized as particles coated with alginate and chitosan, or with glutathione as an additive. The legend in the figure uses the following abbreviations for detailing the compositions: VC denotes vitamin C; CH denotes chitosan; AL denotes alginate; PEDGA denotes poly(ethylene glycol) diacrylate; PEGMA denotes poly(ethylene glycol) methacrylate; and “GXXX” denotes glutathione, with the concentration (μM) indicated by the number “XXX” as shown. The particles were prepared as described in the examples.

FIG. 21A shows representative data demonstrating that hydrogen peroxide present at concentrations of 200-400 μM kills LECs but not APRE-19 cells. FIG. 21B shows representative data that shows that vitamin C is toxic to LECs and ARPE-19 cells at physiological concentrations (1000-2000 μM) found in the vitreous humor.

FIG. 22 shows the proposed concentration gradient of vitamin C in the vitreous humor.

FIG. 23A shows representative data demonstrating that a low concentration of vitamin C can reduce ROS activity induced by hydrogen peroxide, but only over a short-term. FIG. 23B shows representative data demonstrating that ROS activity of LECs increased with the addition of hydrogen peroxide but remained similar to control when treated with 1000 μM of vitamin C for 24 hours. FIG. 23C shows that the ROS activity of APRE-19 did not change with the addition of hydrogen peroxide and did not return to the normal control level when treated with 1000 μM of vitamin C for 24 hours.

FIG. 24 shows representative data demonstrating that encapsulating vitamin C in hydrogels or particles slightly improved its stability. Chitosan-alginate-chitosan particles provided the best protection for vitamin C. Markers are bigger than the error bars (n=4). VC: vitamin C; PEGDA: poly(ethylene glycol) diacrylate; PEGMA: poly(ethylene glycol) methacrylate; CH: chitosan; AL: alginate; GE: gelatin.

FIG. 25 shows representative data demonstrating that glutathione (G) effectively improved vitamin C remaining for at least 15 days in a concentration-dependent manner.

FIG. 26 shows representative data that demonstrates that glutathione is not toxic to LECs and ARPE-19 cells, even at a high concentration of 10000 μM.

Additional advantages of the disclosure can be set forth in part in the description which follows, and in part can be obvious from the description, or can be learned by practice of the disclosure. The advantages of the disclosure can be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a hydrogel,” “a HEMA monomer,” or “a polymer,” includes, but is not limited to, two or more such hydrogels, HEMA monomers, or polymers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, “ascorbic acid” and “vitamin C” can be used interchangeably and refer to a compound having structure represented by the formula:

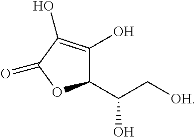 [SMILES: O=C1O[C@H]([C@@H](O)CO)C(O)=C1O]

The use of either term, ascorbic acid or vitamin C, is inclusive of salts thereof, including pharmaceutically acceptable salts. The term ascorbic acid or Vitamin C is inclusive also of all pharmaceutically acceptable derivatives. For example, ascorbic acid can include any of the common mineral salts of ascorbic acid such as sodium ascorbate, which is a compound having a structure represented by the formula:

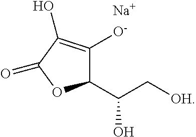 [SMILES: O=C1O[C@H]([C@@H](O)CO)C([O-])=C1O ; [Na+]]

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the terms “optional” or “optionally” means that the subsequently described event or circumstance can or cannot occur, and that the description includes instances where said event or circumstance occurs and instances where it does not.

The following abbreviations are used herein throughout:

APS: Ammonium persulfate

DHA: Docosahexaenoic acid

DMEM: Dulbecco's Modified Eagle's Medium

DPBS: Dulbecco's phosphate-buffered saline

EPA: Eicosapentaenoic acid

FTIR: Fourier transform infrared spectroscopy

HEMA: 2-Hydroxyethyl methacrylate

PEGDA: poly(ethylene glycol) diacrylate

PEGMA: Poly(ethylene glycol) methacrylate

TEMED: N,N,N′,N′-Tetramethylethylenediamine

Unless otherwise specified, temperatures referred to herein are based on atmospheric pressure (i.e. one atmosphere).

### Clinical Context

The eye is susceptible to oxidative damage from free radicals due to its constant exposure to light and very high metabolic activity (Wong-Riley M, Eye Brain, 2010 2:99-116). Several ocular structures contain high levels of antioxidants (e.g. Vitamin C, Vitamin E, glutathione) to protect against damage. However, both age-related degeneration of ocular tissues and ophthalmic surgeries lead to depletion of these antioxidants, resulting in vision-threatening diseases (Holekamp, Am J Ophthalmol. 2010, 149, 32-36). Currently, there are no treatment methods that are capable of locally releasing antioxidants to prevent these diseases and surgical complications. The scientific challenge is protecting the delivered antioxidants from degradation and sustaining local release. To address this challenge, disclosed herein are compositions and methods for permanent, injectable vitreous substitute that serves as a drug delivery reservoir to enable localized and sustained delivery of antioxidants inside the eye. By delivering therapeutics to the lens and other ocular structures, ocular health and function can be dramatically improved following vitrectomy without relying on patient compliance.

It is known that age-related deterioration of the vitreous humor is a major risk factor for retinal detachment and other vision-threatening ocular pathologies. Retinal detachment causes retinal cell death and partial blindness; they can spread if not quickly repaired, ultimately leading to complete blindness. This is normally treated by pars plana vitrectomy in which the natural vitreous is surgically removed and replaced with a temporary substitute that necessitates later removal in a secondary surgical procedure. The success of this procedure often requires patients to lie face-down for up to two weeks to prevent retinal detachments and leads to cataract formation within two years in >95% of patients. Cataract extraction is an additional surgical procedure, with associated costs, pain, and reduced visual acuity (C. J. Siegfried, et al., Invest Ophthalmol Vis Sci. 2017, 58, 4003-4014; Brodie F L, et al, Clin Ophthalmol, 2016 10:955-60; and Chang J S, Smiddy W E, Ophthalmology, 2014 121(9):1720-6).

The most common long-term vitreous substitute, silicone oil, is known to cause several blindness-causing ocular diseases and complications, including cataract, increased intraocular pressure (IOP) (a risk factor for glaucoma), retinal degeneration, and decreased choroidal thickness. Silicone oil emulsification causes proliferative vitreoretinopathy, secondary glaucoma, and keratopathy. Silicone oil also renders ultrasound-based diagnosis of retinal detachment impossible. Further, depending on the location of the retinal tear, patients may be subjected to uncomfortable postoperative positioning, leading to poor compliance and further retinal detachment. Patients unable to comply, often elderly or disabled, forgo surgery or receive more invasive treatments such as a scleral buckle, resulting in further complications (Brodie F L, et al, Clin Ophthalmol, 2016 10:955-60). As a result, silicone oil must be removed within several months, after which the eye fills with liquid aqueous humor (Chang J S, Smiddy W E, Ophthalmology, 2014 121(9):1720-6).

One major challenge is that vitrectomy itself causes increased occurrence of cataract, ocular hypertension, and open-angle glaucoma (Federman J L, Schubert H D, Ophthalmology, 1988 95(7):870-6). These diseases are caused by oxidative damage resulting from increased oxygen levels in the vitreous cavity after surgery. The lens and surrounding area are normally hypoxic, and a high concentration of ascorbic acid (Vitamin C) is required to consume oxygen. After vitrectomy, the homeostatic oxygen gradient is disrupted as the rate of ascorbic acid generation is overtaken by the increased rate of oxygen transport, resulting in cataract and glaucoma. Alterations in the outflow of aqueous humor after removal of the vitreous and oxidative damage to the trabecular meshwork may also lead to elevated TOP, resulting in open-angle glaucoma (C. J. Siegfried, et al., Invest Ophthalmol Vis Sci. 2017, 58, 4003-4014). These diseases that cause serious threats to vision and even blindness have recently been connected to vitrectomy, yet no alternative therapeutic strategies have been explored. The gel-like nature of the natural vitreous slows oxygen diffusion, whereas in the age-related liquefied state, or after removal, there are increased oxygen and depleted ascorbic acid levels in the eye (N. M. Holekamp, Am J Ophthalmol. 2010, 149, 32-36). A hydrogel vitreous substitute could mitigate these issues by retarding intraocular oxygen transport more effectively than a liquid or gas substitute to prevent oxidative damage and could eliminate the need for postoperative patient positioning. Incorporating an antioxidant, such as ascorbic acid, has the potential to further mitigate oxidative damage, potentially preventing cataract or glaucoma resulting from vitrectomy.

Another significant unaddressed challenge is that no permanent vitreous substitutes are currently available. No new substitutes have been introduced to the market since the FDA approved silicone oil as a vitreous substitute in 1994. There is a major clinical need to replace gas and oil substitutes, which would remove the need for postoperative positioning, reduce vision-threatening complications, and eliminate the need for a secondary surgery for substitute removal. The biggest need is in cases of inferior retinal detachment. Since silicone oil and gases are less dense than water, reapproximation of the retina is reliant on patient positioning, requiring an inverted patient position (head down) for up to two weeks. Silicone oil also induces refractive error and increases risks for developing cataract and glaucoma (Federman J L, Schubert H D, Ophthalmology, 1988 95(7):870-6; and Shah M A, et al, Pak J Ophthalmol, 2017 33(2):74-8).

Current vitreous substitutes do not have the viscoelastic and physicochemical properties of the natural vitreous. Overcoming this difficulty will enable better treatments of retinal detachments with vitreous substitutes that fulfil properties imparted by the native vitreous gel. Using a hydrogel also has the potential to eliminate the need for postoperative patient positioning, improving patient compliance and retinal reattachment outcomes. Currently, only an estimated 18-33% of patients comply with postoperative positioning (Brodie F L, et al, Clin Ophthalmol, 10:955-60, 2016), and some patients are physically unable to comply. Swelling properties of the material can be tailored to exert a slight osmotic pressure to reattach the retina without relying on patient compliance. Further, simply having an intact gel in the ocular cavity may help protect the lens after surgery due to decreased convective oxygen transport from the retina (N. M. Holekamp, Am J Ophthalmol. 2010, 149, 32-36). Developing a permanent vitreous substitute will also eliminate the need for a second surgical procedure (and associated costs) for removal (Federman J L, Schubert H D, Ophthalmology, 1988 95(7):870-6).

### Ophthalmological Compositions

In various aspects, the present disclosure pertains to an ophthalmological composition comprising a disclosed vitreous substitute composition, wherein the vitreous substitute composition comprises a gel having the physical properties described herein and a therapeutic agent. In a further aspect, the present disclosure pertains to an ophthalmological composition comprising a disclosed vitreous substitute composition, wherein the vitreous substitute composition comprises a gel having the physical properties described herein and a therapeutic agent, wherein the therapeutic agent is a disclosed antioxidant.

### Vitreous Substitutes

In various aspects, the present disclosure provides a vitreous substitute comprising a gel and at least one antioxidant, wherein the vitreous substitute has physical properties that substantially mimic the same properties of the natural vitreous humor of a human or another animal. In some aspects, the disclosed vitreous substitute is defined by having a loss tangent of less than 1 (for example a loss tangent ranging from about 0.1 to about 0.5) and a refractive index from about 1.33 to about 1.34 (for example a refractive index from about 1.331 to about 1.339 or from about 1.334 to about 1.337). In other embodiments, the vitreous substitute is defined by having a refractive index of less than about 1.4.

In some aspects, the disclosed vitreous substitute can have a storage modulus from about 0.1 Pa to about 1000 Pa, for example from about 1 Pa to about 100 Pa. In some aspects, the disclosed vitreous substitute can have a loss modulus from about 0.01 Pa to about 1000 Pa, for example from about 0.1 Pa to about 100 Pa or from about 0.1 Pa to about 50 Pa.

In some aspects, the vitreous substitute may have a density ranging from about 1.005 g/cm³ to about 1.009 g/cm³.

In some aspects, the vitreous substitute has a transparency of about 75% to about 100% in the electromagnetic radiation in the visible light range. In some embodiments, the vitreous substitute is at least partially transparent to electromagnetic radiation in the near-infrared range. In some embodiments, the vitreous substitute is at least partially transparent to electromagnetic radiation in the ultraviolet or infrared range. In some embodiments, the vitreous substitute is not transparent to electromagnetic radiation in the ultraviolet or infrared range.

In some embodiments, the vitreous substitute may demonstrate shear thinning, i.e., shows a substantial decrease in viscosity with shear rate.

In some embodiments, the vitreous substitute is defined by a diffusion rate ranging from about 0.1×10⁶ cm²/s to about 50×10⁶ cm²/s, for example from about 1×10⁶ cm²/s to about 5×10⁶ cm²/s or from about 2×10⁶ cm²/s to about 4×10⁶ cm²/s.

In some aspects, the gel as used in the vitreous substitute comprises a hydrogel. In some aspects, the vitreous substitute has a water content of greater than 90% by weight, for example greater than 95% by weight, based on the total weight of all components in the vitreous substitute.

In various aspects, the disclosed vitreous substitutes comprise a hydrogel. In some embodiments, the hydrogel comprises a polymer composition, for example a homopolymer, a copolymer, or combinations thereof. In some instances, the hydrogel comprises a copolymer. The copolymer, in some aspects, can reversibly shear thin upon injection to reform a cohesive hydrogel with optical and mechanical properties similar to the natural vitreous humor. In other embodiments, the hydrogel may instead form upon injection by other techniques such as, for example, disulfide bonding, a thermal transition, or self-assembly. In further aspects, the disclosed hydrogels can be tailored in terms of swelling properties. The disclosed hydrogels, can, prior to injection, be purified via dialysis to remove toxic monomers in order to improve biocompatibility.

In some embodiments, the hydrogel as found in the disclosed vitreous substitutes comprises one or more hydrophilic polymers. A hydrophilic polymer may be defined as a polymer having at least 0.1 wt % solubility in water, for example having at least 0.5 wt % solubility. In some embodiments, the hydrophilic polymer has a solubility of at least 1 mg/mL in water.

In some embodiments, the polymer composition comprises one or more vinyl alcohol residues. In some embodiments, the polymer composition comprises one or more acrylamide residues. In some embodiments, the polymer composition may comprise one or more residues selected from a polyethylene glycol derivative or a functionalized polyethylene glycol. In some embodiments, the polymer composition may comprise one or more acrylate residues or one or more methacrylate residues. In some embodiments, the polymer composition may comprise one or more residues selected from acrylamide, N-ornithine acrylamide, N-(2-hydroxypropyl)acrylamide, hydroxyethylacrylate, hydroxyethylmethacrylate, polyethyleneglycol acrylates, polyethylene glycol methacrylates, N-vinylpyrrolidone, N-phenylacrylamide, dimethylaminopropyl methacrylamide, acrylic acid, benzylmethacrylamide, methylthioethylacrylamide, or combinations thereof.

In some aspects, a disclosed vitreous substitute is a hydrogel comprising a copolymer. The copolymer can comprise residues derived from HEMA, PEGDA, and/or PEGMA as described herein.

In some aspects, the disclosed hydrogels comprise a polymer prepared utilizing one or more of: 2-hydroxyethyl methacrylate (HEMA) and/or poly(ethylene glycol) methacrylate (PEGMA). The polymer HEMA has been successfully used in ophthalmic devices such as contact lenses; however, HEMA has not been previously explored as a vitreous substitute since it was evaluated as a pre-formed non-injectable implant. Without wishing to be bound by a particular theory, it is believed that blending HEMA with other hydrophilic monomers or polymers such as PEGMA can add clarity and tailorable swelling properties to the gel.

In other aspects, the disclosed hydrogels comprise a copolymer prepared utilizing one or more of the following monomers: 2-hydroxyethyl methacrylate (HEMA) and/or poly(ethylene glycol) methacrylate (PEGMA). In a further aspect, the copolymer can be prepared utilizing a cross-linking agent, e.g., poly(ethylene glycol) diacrylate (PEGDA) crosslinker.

In a further aspect, disclosed hydrogels can be prepared by free radical polymerization of HEMA, PEGMA, and PEGDA. Briefly, HEMA:PEGMA copolymer hydrogels can be polymerized in water and crosslinked with PEGDA. Ammonium persulfate and N,N,N′,N′-Tetramethylethylenediamine are used to initiate and catalyze the reaction. In a particular aspect, 8.5:6.3:1 molar ratios of HEMA:PEGMA (MW 360):PEGDA (MW 575) can be synthesized and produced clear, soft gels that shear thin and are easily injectable through a small gauge needle without compromising viscoelasticity, as evidenced by the storage (G′) and loss moduli (G″) before and after injection (e.g., see Example 2). In some instances, the disclosed methods of making a disclosed hydrogel comprise steps as described in the Examples herein, as described in published protocols (A. Zellander, et al., PloS one. 2014, 9, e96709), in modifications of published protocols, including those described herein, and method optimization thereof as in keeping with the spirit and scope of the present disclosure.

In various aspects, the disclosed hydrogel is a polymer comprising one or more PEGDA residues. A disclosed hydrogel comprising a polymer comprising one or more PEGDA residues can be formed using the described methods in which polymerization is carried out using PEGDA monomers at a concentration of greater than or equal to about 1 wt % and less than or equal to about 5 wt %. In a further aspect, a disclosed hydrogel comprising a polymer comprising one or more PEGDA residues can be formed using the described methods in which polymerization is carried out using PEGDA monomers at a concentration of greater than or equal to about 1 wt % and less than or equal to about 4 wt %. In a still further aspect, a disclosed hydrogel comprising a polymer comprising one or more PEGDA residues can be formed using the described methods in which polymerization is carried out using PEGDA monomers at a concentration of greater than or equal to about 1.5 wt % and less than or equal to about 4 wt %. In a yet further aspect, a disclosed hydrogel comprising a polymer comprising one or more PEGDA residues can be formed using the described methods in which polymerization is carried out using PEGDA monomers at a concentration of greater than or equal to about 1.5 wt % and less than or equal to about 3.5 wt %. In an even further aspect, a disclosed hydrogel comprising a polymer comprising one or more PEGDA residues can be formed using the described methods in which polymerization is carried out using PEGDA monomers at a concentration of greater than or equal to about 2 wt % and less than or equal to about 3 wt %. In other embodiments, a disclosed hydrogel comprising a polymer composition comprising one or more PEGDA residues can be formed using the described methods in which polymerization is carried out using PEGDA monomers at a concentration ranging from about 0.5 wt % to about 10 wt %, for example from about 1 wt % to about 5 wt %.

In some embodiments, each of the one or more PEDGA residues may independently have a molecular weight of from about 100 to about 10000. In some embodiments, each of the one or more PEGDA residues may have a molecular weight of from about 100 to about 1000. In some embodiment, each of the one or more PEGDA residues have a molecular weight of from about 100 to about 1000, from about 200 to about 1000, from about 300 to about 1000, from about 400 to about 1000, from about 500 to about 1000, from about 600 to about 1000, from about 700 to about 1000, from about 800 to about 1000, from about 900 to about 1000, from about 100 to about 900, from about 200 to about 900, from about 300 to about 900, from about 400 to about 900, from about 500 to about 900, from about 600 to about 900, from about 700 to about 900, from about 800 to about 900, from about 100 to about 800, from about 200 to about 800, from about 300 to about 800, from about 400 to about 800, from about 500 to about 800, from about 600 to about 800, from about 700 to about 800, from about 100 to about 700, from about 200 to about 700, from about 300 to about 700, from about 400 to about 700, from about 500 to about 700, from about 600 to about 700, from about 100 to about 600, from about 200 to about 600, from about 300 to about 600, from about 400 to about 600, from about 500 to about 600, from about 100 to about 500, from about 200 to about 500, from about 300 to about 500, from about 400 to about 500, from about 100 to about 400, from about 200 to about 400, from about 300 to about 400, from about 100 to 300, from about 200 to 300, or from about 100 to 200.

In various aspects, the disclosed hydrogel is a polymer comprising one or more PEGMA residues. A disclosed hydrogel comprising a polymer comprising one or more PEGMA residues can be formed using the described methods in which polymerization is carried out using PEGMA monomers at a concentration of greater than or equal to about 3 wt % and less than or equal to about 8 wt %. In a further aspect, a disclosed hydrogel comprising a polymer comprising one or more PEGMA residues can be formed using the described methods in which polymerization is carried out using PEGMA monomers at a concentration of greater than or equal to about 4 wt % and less than or equal to about 8 wt %. In a still further aspect, a disclosed hydrogel comprising a polymer comprising one or more PEGMA residues can be formed using the described methods in which polymerization is carried out using PEGMA monomers at a concentration of greater than or equal to about 5 wt % and less than or equal to about 8 wt %. In a yet further aspect, a disclosed hydrogel comprising a polymer comprising one or more PEGMA residues can be formed using the described methods in which polymerization is carried out using PEGMA monomers at a concentration of greater than or equal to about 5 wt % and less than or equal to about 7 wt %. In an even further aspect, a disclosed hydrogel comprising a polymer comprising one or more PEGMA residues can be formed using the described methods in which polymerization is carried out using PEGMA monomers at a concentration of greater than or equal to about 5.5 wt % and less than or equal to about 7.5 wt %. In a still further aspect, a disclosed hydrogel comprising a polymer comprising one or more PEGMA residues can be formed using the described methods in which polymerization is carried out using PEGMA monomers at a concentration of greater than or equal to about 6 wt % and less than or equal to about 7 wt %. In other embodiments, a disclosed hydrogel comprising a polymer composition comprising one or more PEGMA residues can be formed using the described methods in which polymerization is carried out using PEGMA monomers at a concentration ranging from about 0.5 wt % to about 10 wt %, for example from about 1 wt % to about 5 wt %.

In some embodiments, each of the one or more PEGMA residues may independently have a molecular weight from about 100 to about 8000, for example from about 100 to about 4000. In some embodiments, each of the one or more PEGMA residues have a molecular weight of from about 100 to about 500. In some embodiments, each of the one or more PEGMA residues have a molecular weight of from about 100 to about 500, from about 150 to about 500, from about 200 to about 500, from about 250 to about 500, from about 280 to about 500, from about 300 to about 500, from about 380 to about 500, from about 400 to about 500, from about 450 to about 500, from about 100 to about 450, from about 150 to about 450, from about 200 to about 450, from about 250 to about 450, from about 280 to about 450, from about 300 to about 450, from about 380 to about 450, from about 400 to about 450, from about 100 to about 400, from about 150 to about 400, from about 200 to about 400, from about 250 to about 400, from about 280 to about 400, from about 300 to about 400, from about 380 to about 400, from about 100 to about 380, from about 150 to about 380, from about 200 to about 380, from about 250 to about 380, from about 280 to about 380, from about 300 to about 380, from about 100 to about 300, from about 150 to about 300, from about 200 to about 300, from about 250 to about 300, from about 280 to about 300, from about 100 to about 280, from about 150 to about 280, from about 200 to about 280, from about 250 to about 280, from about 100 to about 250, from about 150 to about 250, from about 200 to about 250, from about 100 to 200, from about 150 to 200, or from about 100 to 150.

In various aspects, the disclosed hydrogel is a copolymer comprising PEGDA and PEGMA residues. A disclosed hydrogel comprising a polymer comprising PEGDA and PEGMA residues can be formed using the described methods in which polymerization is carried out using PEGDA and PEGMA monomers each at a concentration of greater than or equal to about 2.5 wt % and less than or equal to about 4 wt %. In a further aspect, a disclosed hydrogel comprising a polymer comprising PEGDA and PEGMA residues can be formed using the described methods in which polymerization is carried out using PEGDA and PEGMA monomers each at a concentration of greater than or equal to about 3 wt % and less than or equal to about 3.8 wt %. In some instances, the foregoing copolymer can comprise HEMA, in which HEMA is present in the polymerization reaction at a concentration of from about 0.1 wt % to about 1.0 wt %.

In various aspects, a disclosed hydrogel can comprise a polymer formed from one or more 2-hydroxyethylmethacrylate (HEMA) residues and one or more acrylamide residues; one or more HEMA residues and one or more poly(ethylene glycol)methacrylate (PEGMA) residues; one or more HEMA residues and one or more methacrylic acid residues; one or more HEMA residues and one or more poly(vinyl alcohol) (PVA) residues; or one or more PVA and one or more acrylamide residues. In some embodiments, the disclosed hydrogel can be further formed from a disulfide cross-linker such as bisacryloylcystamine.

In order to improve biocompatibility, gels can be dialyzed against deionized water. After dialysis, the formulation can be injected or freeze-dried for storage at room temperature in dry form. Freeze-dried polymers can be rehydrated in aqueous solutions, including balanced salt solutions at physiological, including, but not limited to a pH of about 7.4. In various aspects, an aqueous solution used for rehydration can comprise a pharmaceutically acceptable buffer. For intraocular analysis, gels can be sterilized and will self-assemble in the eye upon injection (Uesugi K, et al, Invest Ophthalmol Vis Sci, 2017 58(10):4068-75; and K. E. Swindle, P. D. Hamilton, N. Ravi, J. Biomed. Mater. Res. A. 2008, 87, 656-665).

In various aspects, the hydrogels disclosed herein can gel, either in the presence or absence of a disclosed antioxidant, over a period of from about 15 minutes to about 72 hours. In a further aspect, the gelling time can be from about 30 minutes to about 24 hours.

The disclosed vitreous substitute can comprise a first hydrogel, in which the first hydrogel is comprising HEMA, PEGDA, and/or PEGMA residues as disclosed herein, a second hydrogel, and one or more disclosed antioxidant. The second hydrogel can be any suitable hydrogel as known to the skilled artisan, including, but not limited to a hydrogel disclosed in U.S. Pat. Appl. Nos. 20050208102, 20050074497, 20090252781, 20140296158, 20130123195, 20150250891, 20160331738, 20160331738, 20170112888, 20180280688, 20180045978, and 20180200340; and in U.S. Pat. Nos. 5,522,888, 5,716,633, 7,939,579, 9,125,807, 9,205,181, 9,775,906, 9,987,367, and 10251954. In some instances, the first hydrogel concentration is essentially about 0 wt %. In other instances, the second hydrogel concentration is essentially about 0 wt %. Representative examples of the second hydrogel as may be used in the disclosed vitreous substitute include, but are not limited to, hyaluronic acid, collagen, gellan, silk, fibrin, alginate, chitosan, polyacrylamide and methacrylate derivatives thereof, polyacrylic acid and methacrylate derivatives thereof, polyvinyl alcohol, polyethylene glycol and derivatives thereof, polypropylene glycol and derivatives thereof, polymerized ascorbic acid, or combinations thereof.

In some embodiments, the vitreous substitute may comprise one or more thermogelling agents, such as for example poloxamers.

### Antioxidants

In various aspects, any suitable antioxidant can be used as a therapeutic agent in the disclosed vitreous substitutes. As used herein, it should be understood that the use of the term “antioxidant” is inclusive of free-radical scavengers and can be used interchangeably with “free-radical scavenger.” The term “free-radical scavenger” as used herein refers to a substance, such as an antioxidant, that helps protect cells from the damage caused by free radicals.

In some embodiments, the antioxidant is present in an amount sufficient to produce a therapeutic effect without showing any significant toxicity to the tissues of the eye.

In some aspects, the antioxidant used can comprise vitamin A; vitamin C (ascorbic acid); N-acetylcysteine; glutathione; a zinc compound; a copper compound; vitamin E and derivatives thereof, including, but not limited to, alpha, beta, gamma, and delta tocopherol and/or alpha, beta, gamma, and delta tocotrienols, and derivatives thereof; selenous acid; sodium selenite; a saturated and unsaturated fatty acid, including, but not limited to, 6-O-lauroyl ascorbate, 6-O-myristoyl ascorbate, 6-O-oleoyl ascorbate, 6-O-palmitoyl ascorbate, 6-O-linoleoyl ascorbate, 6-O-stearoyl ascorbate; 1-carnitine and derivatives such as 1-carnitine acetate; retinal; tretinoin; timolol; lutein; thyroxine; pyrroloquinolone; probucol; captopril; uric acid; erithorbic acid and its salts; α-lipoic acid; hydralazine; gallic acid; lycopene; astaxanthin; zeaxanthin; ferulic acid; quercetin; eugenol; isoeugenol; melatonin; resveratrol; mannitol; trolox; methylethylpiridinol; taufon; a thiol antioxidant; beta carotene; and combinations of one or more of the foregoing.

In a further aspect, the antioxidant used can comprise vitamin E; vitamin C (ascorbic acid); lutein; zeaxanthin; a zinc compound; a copper compound; beta carotene; one or more omega-3 fatty acid, e.g., DHA or EPA; or combinations thereof. That is, one or more of the components known for use in AREDS or AREDS2 compositions.

In some embodiments, the antioxidant used can comprise alpha lipoic acid, riboflavin, taurine, uric acid, tyrosine, transferring, selenium, zinc, superoxide dismutase, glutathione peroxidase, catalase, pigment epithelium-derived factor (PEDF), or combinations thereof. In some embodiments, the antioxidant can be present in a concentration that mimics the normal concentration of the antioxidant as found in the vitreous of a human or animal; representative examples of such concentrations are found in Ankamah, E. et al. “Vitreous Antioxidants, Degeneration, and Vitreo-Retinopathy: Exploring the Links” Antioxidants 2020, 9,7, doi:10,3390/antiox901007, incorporated herein by reference in its entirety.

In a further aspect, a thiol antioxidant can be selected from glutathione (GSH), oxidation-type glutathione or oxidized glutathione (GSSG), N-acetylcysteine, thioctic acid, 2-oxo-thiazolidine-4-carboxylic acid, cysteine, glutamylcysteine, ethanethiol, 1,4-butanethiol, 2-mercaptoethylether, pentaerythretoltetrathiopropionate and acetate, polyethyleneglycolimercaptoacetate and methylthioglycolate, allyl mercaptan, 2-mercaptoethanol, 3-mercaptopropanol, 4-mercaptobutanol, 1-thioglycerol, thioerythritol, 2,3-dimercaptopropanol, pentaerythretolmono (di; tri)thiopropionate or acetate, thioglycolic acid, thioacetic acid, 3-mercaptopropionic acid, thiolactic acid, thiomalic acid, thiosuccinic acid, thiosalicylic acid, thiobenzoic acid and their respective water soluble salts, furfuryl mercaptan, 2-mercaptobenzimidazole, 2-mercaptobenzoxazole, 2-mercapto-3-pyridinol, dimethylaminopropanethiol, 2-mercaptoethylamine, 2-n-butylaminoethanethiol; derivatives of the foregoing; and mixtures of the foregoing or in combination with another disclosed antioxidant thereof.

In a further aspect, a thiol antioxidant can be selected from N-acetylcysteine, thioctic acid, 2-oxo-thiazolidine-4-carboxylic acid, cysteine, glutamylcysteine and mixtures thereof.

In a further aspect, a thiol antioxidant can be selected from GSH, ophthalmically acceptable salts of GSH, GSSG, ophthalmically acceptable salts of GSSG, precursors thereof and mixtures thereof. In a still further aspect, a thiol antioxidant can be selected from GSH, GSSG, ophthalmically acceptable salts thereof and mixtures thereof. In a yet further aspect, a thiol antioxidant can be selected from GSH, GSSG and mixtures thereof. In an even further aspect, a thiol antioxidant comprises GSH.

In a further aspect, ophthalmically acceptable anions included in the ophthalmically acceptable salts of an antioxidant include chloride, bromide, iodide, sulfate, bisulfate, phosphate, acid phosphate, nitrate, acetate, maleate, fumarate, oxalate, lactate, tartrate, citrate, gluconate, saccharate, p-toluene sulfonate and the like. Ophthalmically acceptable derivatives useful as an antioxidant include esters, acids and the like.

In other aspects, the antioxidant present in a disclosed vitreous substitute can be one or more of an agent selected from ascorbic acid, Na ascorbate, K ascorbate, Ca ascorbate, Mg ascorbate, Zn ascorbate; 6-O-esters of ascorbic acid with C2 to C20 straight, branched, saturated and unsaturated fatty acids: 6-O-lauroyl ascorbate, 6-O-myristoyl ascorbate, 6-O-oleoyl ascorbate, 6-O-palmitoyl ascorbate, 6-O-linoleoyl ascorbate, 6-O-stearoyl ascorbate; 6-O-ester of ascorbic acid with d, or dl-α-tocopheryl hemisuccinate; 6-O-esters of ascorbic acid with reduced glutathione and d, or dl-α-tocopherols; reduced glutathione and glutathione ester of reduced glutathione with d or dl-α-tocopherol; d and dl-tocopherol (α, β, γ, δ isomers) and the acetate, hemisuccinate, nicotinate, and succinate-PEG ester (TPGS) derivatives of the foregoing tocopherol isomers; superoxide dismutase; β-carotene; melatonin; trans resveratrol; trolox; coenzyme Q; catalase; various peroxidases; cysteine, ester of cysteine with ethanol, HCl salt of the ester of cysteine with ethanol, the salt of ascorbic acid with the ester of cysteine with ethanol, the d or dl-α-tocopherol-hemisuccinate salt of the ester of cysteine with ethanol, the ester of cysteine with d, or dl-α-tocopherol, N-acetylcysteine, Na, K, Ca, Mg, Zn salts of N-acetylcysteine, ester of N-acetyl cysteine with ethanol or d, or dl-α-tocopherol; 1-carnitine; 1-carnitine acetate; retinal; tretinoin; timolol; lutein; thyroxine; pyrroloquinolone; probucol; captopril; desferal Mn+3; uric acid; erithorbic acid and its salts; α-lipoic acid; lycopene; astaxanthin; zeaxanthin; ferulic acid; quercetin; eugenol and isoeugenol; prostaglandins; latanoprost, bimatoprost, travoprost; (−)-epicatechin; (−)-epigallocatechin gallate; butylated hydroxytoluene; butylated hydroxyanisole; rutinal; fisetin; sulfite and bisulfite salts (Na, K, Ca, Mg). In some embodiments, the antioxidant may comprise L-ascorbic acid, ascorbic acid 6-palmitate, or combinations thereof.

In some aspects, the antioxidant present in the disclosed vitreous substitute may comprise one or more of the ascorbic acid derivatives described in Macan, A. et al. “Therapeutic Perspective of Vitamin C and Its Derivatives” Antioxidants 2019, 8, 247, doi:10.3390/antiox8080247, incorporated herein by reference in its entirety for all purposes.

In various aspects, the antioxidant can be present in a disclosed vitreous substitute at a concentration of from about 0.001 ng/ml to about 100 mg/ml; about 0.001 ng/ml to about 10 mg/ml; about 0.001 ng/ml to about 1 mg/ml; about 0.01 ng/ml to about 100 mg/ml; about 0.01 ng/ml to about 10 mg/ml; about 0.01 ng/ml to about 1 mg/ml; about 0.1 ng/ml to about 100 mg/ml; about 0.1 ng/ml to about 10 mg/ml; about 0.1 ng/ml to about 1 mg/ml; about 1 ng/ml to about 100 mg/ml; about 1 ng/ml to about 10 mg/ml; or a sub-range within the foregoing ranges.

In a further aspect, ascorbic acid, or a suitable salt thereof, can be present in a disclosed vitreous substitute at a concentration of from about 0.001 ng/ml to about 1 mg/ml. In a still further aspect, ascorbic acid, or a suitable salt thereof, can be present in a disclosed vitreous substitute at a concentration of from about 1 μg/ml to about 1000 μg/ml. In a yet further aspect, ascorbic acid, or a suitable salt thereof, can be present in a disclosed vitreous substitute at a concentration of from about 100 μg/ml to about 1000 μg/ml. In an even further aspect, ascorbic acid, or a suitable salt thereof, can be present in a disclosed vitreous substitute at a concentration of from about 200 μg/ml to about 800 μg/ml. In a still further aspect, ascorbic acid, or a suitable salt thereof, can be present in a disclosed vitreous substitute at a concentration of from about 300 μg/ml to about 700 μg/ml. In another aspect, ascorbic acid, or a suitable salt or derivative thereof, may be present in the disclosed vitreous substitute in a concentration of from about 0.1 mM to about 5 mM, for example, from 0.1 mM to about 1 mM.

In a further aspect, a tocopherol, or derivative thereof, can be present in a disclosed vitreous substitute at a concentration of from about 0.001 ng/ml to about 1 mg/ml. In a still further aspect, a tocopherol, or derivative thereof, can be present in a disclosed vitreous substitute at a concentration of from about 1 μg/ml to about 200 μg/ml. In a yet further aspect, a tocopherol, or derivative thereof, can be present in a disclosed vitreous substitute at a concentration of from about 1 μg/ml to about 100 μg/ml. In an even further aspect, a tocopherol, or derivative thereof, can be present in a disclosed vitreous substitute at a concentration of from about 5 μg/ml to about 75 μg/ml. In a still further aspect, a tocopherol, or derivative thereof, can be present in a disclosed vitreous substitute at a concentration of from about 5 μg/ml to about 50 μg/ml.

In a further aspect, a glutathione, e.g., reduced glutathione, or derivative thereof, can be present in a disclosed vitreous substitute at a concentration of from about 0.001 ng/ml to about 1 mg/ml. In a still further aspect, a glutathione, e.g., reduced glutathione, or derivative thereof, can be present in a disclosed vitreous substitute at a concentration of from about 1 μg/ml to about 200 μg/ml. In a yet further aspect, a glutathione, e.g., reduced glutathione, or derivative thereof, can be present in a disclosed vitreous substitute at a concentration of from about 1 μg/ml to about 100 μg/ml. In an even further aspect, a glutathione, e.g., reduced glutathione, or derivative thereof, can be present in a disclosed vitreous substitute at a concentration of from about 5 μg/ml to about 75 μg/ml. In a still further aspect, a glutathione, e.g., reduced glutathione, or derivative thereof, can be present in a disclosed vitreous substitute at a concentration of from about 5 μg/ml to about 50 μg/ml. In some aspects, a glutathione, e.g., reduced glutathione, or a derivative thereof, can be present in a disclosed vitreous substitute at a concentration from about 0.1 mM to about 100 mM, form about 0.05 mM to about 10 mM, from about 1 mM to about 10 mM, from about 2 mM to 10 mM, from about 2 mM to about 4 mM, or from about 4 mM to about 10 mM. In some aspects, a glutathione, e.g., reduced glutathione, or a derivative thereof, can be present in a disclosed vitreous substitute at a concentration of about 1 mM, about 2 mM, about 4 mM, about 10 mM, or more.

In a further aspect, a melatonin, or derivative thereof, can be present in a disclosed vitreous substitute at a concentration of from about 0.001 ng/ml to about 1 mg/ml. In a still further aspect, a tocopherol, or derivative thereof, can be present in a disclosed vitreous substitute at a concentration of from about 1 pg/ml to about 200 pg/ml. In a yet further aspect, a tocopherol, or derivative thereof, can be present in a disclosed vitreous substitute at a concentration of from about 1 pg/ml to about 100 pg/ml. In an even further aspect, a tocopherol, or derivative thereof, can be present in a disclosed vitreous substitute at a concentration of from about 5 pg/ml to about 75 pg/ml. In a still further aspect, a tocopherol, or derivative thereof, can be present in a disclosed vitreous substitute at a concentration of from about 5 pg/ml to about 50 pg/ml.

In some aspects, ascorbic acid can be used as an antioxidant. Ascorbic acid has several desirable characteristics. It is present at a remarkably high level in the vitreous humor (2 mM compared to 50-60 μM in blood; see Y. B. Shui, et al., Arch Ophthalmol. 2009, 127, 475-482). Ascorbic acid solutions have the same effect as all the other antioxidant s found in the vitreous combined, suggesting the potent antioxidant effect of ascorbic acid (Chen-Roetling J, et al, Biochem Biophys Res Commun, 2018 503(1):152-6). It also accounts for 75% of the antioxidant potential in the aqueous humor (C. J. Siegfried, et al., Invest Ophthalmol Vis Sci. 2017, 58, 4003-4014). While there are other factors that affect cataract, ascorbic acid appears to be a significant component that can control regulate oxygen at the lens surface to prevent cataract.

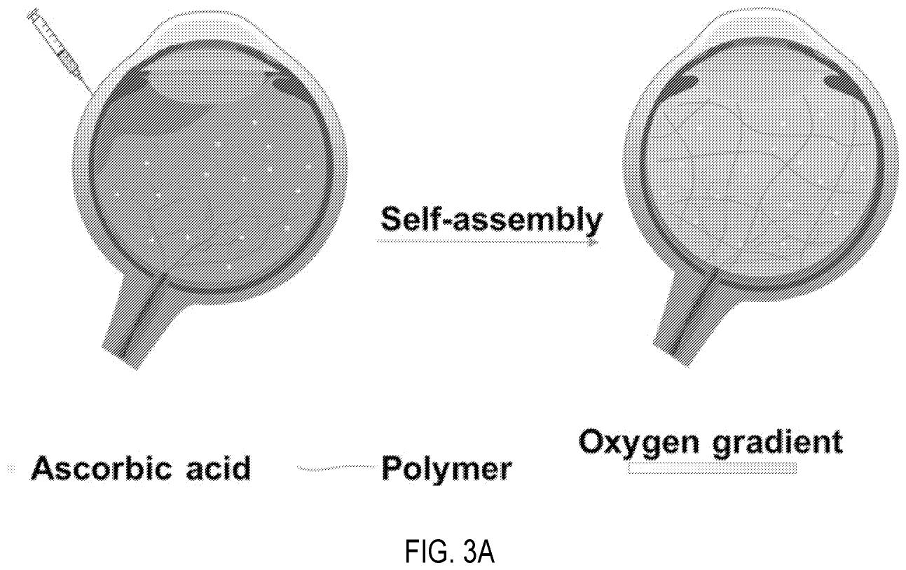

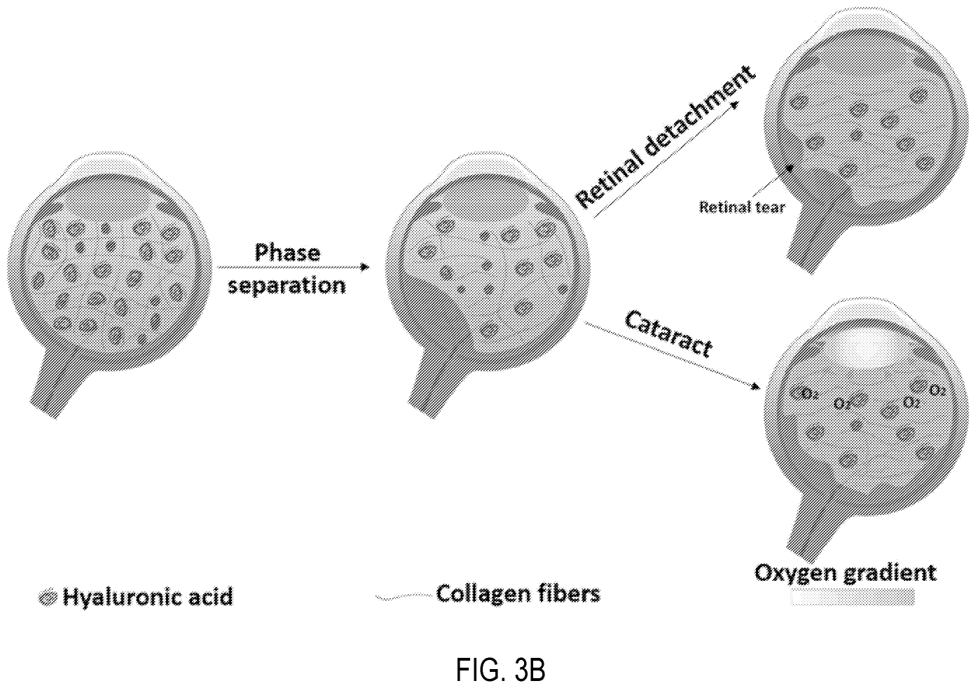

To protect the antioxidant prior to injection and to control release, ascorbic acid can be encapsulated and then blended with the vitreous substitute prior to injection (FIG. 3). Nanoparticles encapsulating ascorbic acid can sustain release from about 0.001 mM to about 100 mM concentration to replicate levels found in the vitreous. Disclosed herein are nanoparticle and hydrogel formulations loaded ascorbic acid in multiple. The encapsulation strategy can facilitate rapid initial release of the antioxidant, which can be desirable for immediate protection of ocular tissues during and after vitrectomy, followed by controlled release to maintain ascorbic acid concentration for approximately 1 month until antioxidant levels are restored in the eye by the ciliary body (Sebag J, The Vitreous: Structure, Function, and Pathobiology, 1989).

In various aspects, encapsulation in rapidly dissolving natural polymers such as gelatin and alginate (Lee E M, et al, J Nanomat, 2014 124:236) can be utilized to protect and stabilize the antioxidant prior to intraocular injection. Alternately, to prevent ascorbic acid oxidation, EDTA can be incorporated into the disclosed hydrogel composition. EDTA is used in ophthalmic formulations (Rao M V L, et al, J Sci Food Agricul, 1959 10(8):436-41), reverse oxidation by ocular enzymes such as thioredoxin reductase (May J M, et al, J Biol Chem, 1997 272:22607-10), or stabilization with retinyl ascorbate (Das N, et al, Eur J Pharm Sci, 2010 41(5):571-88). If ascorbic acid is ineffective at protecting the lens from oxidative stress, other antioxidants can be evaluated such as glutathione, which is highly concentrated in the lens (Wang-Su S T, et al, Invest Ophthalmol Vis Sci, 44:4829-36, 2003), or Vitamin A, Vitamin E, or lutein which are known to protect eye health (Chew E Y, Ophthalmology, 2012 119(11):2282-9; and Zhang J, et al, Biomacromolecules, 2016 17(11):3648-58).

In various aspects, the antioxidant can be encapsulated in particles such as gelatin-alginate nanoparticles, which can be prepared using a water-in-oil emulsification technique with modifications (Lee E M, et al, J Nanomat, 2014:124236, 2014). Briefly, alginate and gelatin can be dissolved in heated water at a 1:2 weight ratio at 0.075 g/mL, and ascorbic acid can be added to the solution. The solution can be added dropwise into rapidly stirring corn oil for 30 min. Particles can be precipitated in acetone, then crosslinked in 1% glutaraldehyde to slow therapeutic release. Particles can then be collected using centrifugation and washed with distilled water. Drug release profile and particle size can be controlled by manipulating the ratio between gelatin:alginate, polymer concentration, crosslinker concentration, and ascorbic acid loading. Particles composed of chitosan, alginate-chitosan, gelatin, and gelatin-alginate in size ranges of 200 nm to 1.5 μm that sustain release for several days to several weeks have been synthesized.

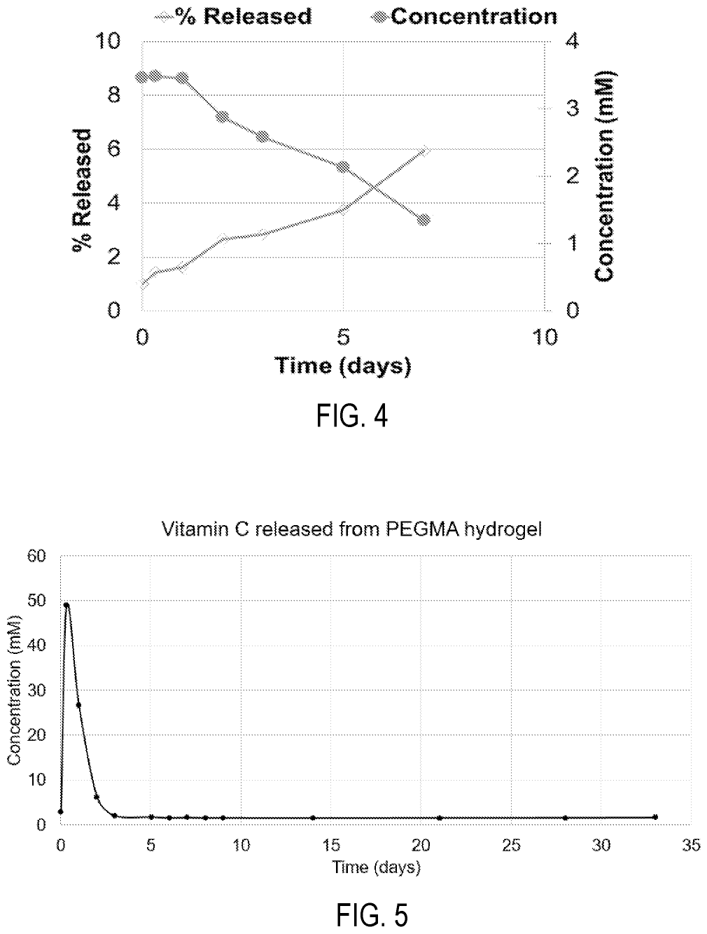

Ascorbic acid loading can be confirmed by measuring absorbance using UV-Vis spectroscopy at 265 nm, or using an appropriate assay system (e.g., commercially available kits such as Ascorbic Acid Assay Kit MAK074 or Ascorbic Acid Assay Kit II MAK075 available from Sigma-Aldrich Corporation, St. Louis, Mo.). Release rate of ascorbic acid from the particles and composite gels can be evaluated by incubating in phosphate buffered saline at 37° C. with shaking. Eluent can be removed and replaced with fresh saline after 1, 6, 12, and 24 hours, then on days 3, 5, 7, 14, 21, and 28. Representative data show initial burst release followed by sustained release for at least 7 days (FIG. 4).

In some aspects, the antioxidant present in a disclosed vitreous substitute can include ascorbic acid in combination with a glutathione, e.g., reduced glutathione (GSH) or a derivative thereof. The further addition of a glutathione with ascorbic acid in the vitreous substitutes disclosed herein can improve the stability of the ascorbic acid as compared to other methods. In some aspects, a glutathione such as reduced glutathione (GSH) may be present, in combination with ascorbic acid, in a disclosed vitreous substitute at a concentration from about 0.01 mM to about 100 mM, from about 0.05 mM to about 10 mM, from about 1 mM to about 10 mM, for example from about 2 mM to about 10 mM, from about 4 mM to about 10 mM, from about 1 mM to about 4 mM, from about 2 mM to about 4 mM, or from about 4 mM to about 10 mM. In some aspects, a glutathione such as reduced glutathione (GSH) may be present, in combination with ascorbic acid, in a disclosed vitreous substitute at a concentration of about 1 mM, of about 2 mM, of about 3 mM, about 4 mM, about 10 mM, or more. In some embodiments, ascorbic acid, or suitable salts or derivatives thereof, may be present in the disclosed vitreous substitutes (when used in combination with a glutathione) in a concentration from about 0.1 mM to about 5 mM, for example, from about 0.1 to about 1 mM. In some embodiments, ascorbic acid, or suitable salts or derivatives thereof, may be present in the disclosed vitreous substitutes (when used in combination with a glutathione) in a concentration of about 0.1 mM, about 0.2 mM, about 0.3 mM, about 0.4 mM, about 0.5 mM, about 0.6 mM, about 0.7 mm, about 0.8 mm, about 0.9 mM, or more.

### Additional Therapeutic Agents

In some embodiments, the vitreous substitute as described in the present disclosure may further comprise one or more additional therapeutic agents.

As used herein, a “therapeutic agent” refers to one or more therapeutic agents, active ingredients, or substances that can be used to treat a medical condition of the eye or a cancer. The therapeutic agents are typically ophthalmically acceptable and are provided in a form that does not cause adverse reactions when the compositions disclosed herein are placed in an eye. As discussed herein, the therapeutic agents can be released from the disclosed compositions in a biologically active form. For example, the therapeutic agents may retain their three-dimensional structure when released from the system into an eye.

It is further understood, that as used herein, the terms “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

In some embodiments, the therapeutic agent may comprise an agent useful in the treatment of an ophthalmological disorder or an eye disease such as: beta-blockers including timolol, betaxolol, levobetaxolol, and carteolol; miotics including pilocarpine; carbonic anhydrase inhibitors; serotonergics; muscarinics; dopaminergic agonists; adrenergic agonists including apraclonidine and brimonidine; anti-angiogenesis agents; anti-infective agents including quinolones such as ciprofloxacin and aminoglycosides such as tobramycin and gentamicin; non-steroidal and steroidal anti-inflammatory agents, such as suprofen, diclofenac, ketorolac, rimexolone and tetrahydrocortisol; growth factors, such as EGF; immunosuppressant agents; and anti-allergic agents including olopatadine; prostaglandins such as latanoprost; 15-keto latanoprost; travoprost; and unoprostone isopropyl.

In some embodiments, the therapeutic agent is selected from the group consisting of an anti-inflammatory agent, a calcineurin inhibitor, an antibiotic, a nicotinic acetylcholine receptor agonist, and an anti-lymphangiogenic agent. In some embodiments, the anti-inflammatory agent may be cyclosporine. In some embodiments, the calcineurin inhibitor may be voclosporin. In some embodiments, the antibiotic may be selected from the group consisting of amikacin, gentamycin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, and tetracycline. In some embodiments, the nicotinic acetylcholine receptor agonist may be any of pilocarpine, atropine, nicotine, epibatidine, lobeline, or imidacloprid. In some embodiments, the anti-lymphangiogenic agent may be a vascular endothelial growth factor C (VEGF-C) antibody, a VEGF-D antibody or a VEGF-3 antibody.

In some aspects, the therapeutic agent may be selected from: a beta-blocker, including levobunolol (BETAGAN), timolol (BETIMOL, TIMOPTIC), betaxolol (BETOPTIC) and metipranolol (OPTIPRANOLOL); alpha-agonists, such as apraclonidine (IOPIDINE) and brimonidine (ALPHAGAN); carbonic anhydrase inhibitors, such as acetazolamide, methazolamide, dorzolamide (TRUSOPT) and brinzolamide (AZOPT); prostaglandins or prostaglandin analogs such as latanoprost (XALATAN), bimatoprost (LUMIGAN) and travoprost (TRAVATAN); miotic or cholinergic agents, such as pilocarpine (ISOPTO CARPINE, PILOPINE) and carbachol (ISOPTO CARBACHOL); epinephrine compounds, such as dipivefrin (PROPINE); forskolin; or neuroprotective compounds, such as brimonidine and memantine; a steroid derivative, such as 2-methoxyestradiol or analogs or derivatives thereo; or an antibiotic.

The term “VEGF” refers to a vascular endothelial growth factor that induces angiogenesis or an angiogenic process, including, but not limited to, increased permeability. As used herein, the term “VEGF” includes the various subtypes of VEGF (also known as vascular permeability factor (VPF) and VEGF-A) that arise by, e.g., alternative splicing of the VEGF-A/VPF gene including VEGF121, VEGF165 and VEGF189. Further, as used herein, the term “VEGF” includes VEGF-related angiogenic factors such as PIGF (placental growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1), VEGFR-2 (KDR/Flk-1), or VEGFR-3 (FLT-4). The term “VEGF” can be used to refer to a “VEGF” polypeptide or a “VEGF” encoding gene or nucleic acid.

The term “anti-VEGF agent” refers to an agent that reduces, or inhibits, either partially or fully, the activity or production of a VEGF. An anti-VEGF agent can directly or indirectly reduce or inhibit the activity or production of a specific VEGF such as VEGF165. Furthermore, “anti-VEGF agents” include agents that act on either a VEGF ligand or its cognate receptor so as to reduce or inhibit a VEGF-associated receptor signal. Non-limiting examples of “anti-VEGF agents” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

In some embodiments, the therapeutic agent may comprise an anti-VEGF agent. Representative examples of anti-VEGF agents include ranibizumab, bevacizumab, aflibercept, KH902 VEGF receptor-Fc, fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-, hydroxyisovalerylshikonin, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, G6-31 antibody, a fusion antibody and an antibody that binds to an epitope of VEGF. Additional non-limiting examples of anti-VEGF agents useful in the present methods include a substance that specifically binds to one or more of a human vascular endothelial growth factor-A (VEGF-A), human vascular endothelial growth factor-B (VEGF-B), human vascular endothelial growth factor-C (VEGF-C), human vascular endothelial growth factor-D (VEGF-D) and human vascular endothelial growth, factor-E (VEGF-E), and an antibody that binds, to an epitope of VEGF.

In various aspects, the anti-VEGF agent is the antibody ranibizumab or a pharmaceutically acceptable salt thereof. Ranibizumab is commercially available under the trademark LUCENTIS. In another embodiment, the anti-VEGF agent is the antibody bevacizumab or a pharmaceutically acceptable salt thereof. Bevacizumab is commercially available under the trademark AVASTIN. In another embodiment, the anti-VEGF agent is aflibercept or a pharmaceutically acceptable salt thereof. Aflibercept is commercially available under the trademark EYLEA. In one embodiment, the anti-VEGF agent is pegaptanib or a pharmaceutically acceptable salt thereof. Pegaptinib is commercially available under the trademark MACUGEN. In another embodiment, the anti-VEGF agent is an antibody or an antibody fragment that binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of the three-dimensional structure of VEGF that is displayed, such that the epitope is exposed on the surface of the folded VEGF molecule. In one embodiment, the epitope is a linear amino acid sequence from VEGF.

In various aspects, the therapeutic agent may comprise an agent that blocks or inhibits VEGF-mediated activity, e.g., one or more VEGF antisense nucleic acids. The present disclosure provides the therapeutic or prophylactic use of nucleic acids comprising at least six nucleotides that are antisense to a gene or cDNA encoding VEGF or a portion thereof. As used herein, a VEGF “antisense” nucleic acid refers to a nucleic acid capable of hybridizing by virtue of some sequence complementarity to a portion of an RNA (preferably mRNA) encoding VEGF. The antisense nucleic acid may be complementary to a coding and/or noncoding region of an mRNA encoding VEGF. Such antisense nucleic acids have utility as compounds that prevent VEGF expression, and can be used in the treatment of diabetes. The antisense nucleic acids of the disclosure are double-stranded or single-stranded oligonucleotides, RNA or DNA or a modification or derivative thereof, and can be directly administered to a cell or produced intracellularly by transcription of exogenous, introduced sequences.

The VEGF antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisense molecules include short double stranded RNAs, known as siRNAs, and short hairpin RNAs, and long dsRNA (>50 bp but usually 500 bp).

In various aspects, the therapeutic agent may comprise one or more ribozyme molecule designed to catalytically cleave gene mRNA transcripts encoding VEGF, preventing translation of target gene mRNA and, therefore, expression of the gene product.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA and must include the well-known catalytic sequence responsible for mRNA cleavage. For this sequence, see, e.g., U.S. Pat. No. 5,093,246. While ribozymes that cleave mRNA at site-specific recognition sequences can be used to destroy mRNAs encoding VEGF, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art. The ribozymes of the present disclosure also include RNA endoribonucleases (hereinafter “Cech-type ribozymes”) such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place. The disclosure encompasses those Cech-type ribozymes that target eight base-pair active site sequences that are present in the gene encoding VEGF.

In further aspects, the therapeutic agent may comprise an antibody that inhibits VEGF such as bevacizumab or ranibizumab. In still further aspects, therapeutic agent may comprise an agent that inhibits VEGF activity such as a tyrosine kinase stimulated by VEGF, examples of which include, but are not limited to lapatinib, sunitinib, sorafenib, axitinib, and pazopanib. The term “anti-RAS agent” or “anti-Renin Angiotensin System agent” refers to refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

In some embodiments, the therapeutic agent may comprise a renin angiotensin system (RAS) inhibitor. In some embodiments, the renin angiotensin system (RAS) inhibitor is one or more of an angiotensin-converting enzyme (ACE) inhibitor, an angiotensin-receptor blocker, and a renin inhibitor.

Non limiting examples of angiotensin-converting enzyme (ACE) inhibitors which are useful in the present invention include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof.

Non limiting examples of angiotensin-receptor blockers which are useful in the present invention include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

Non limiting examples of renin inhibitors which may be used as therapeutic agents include, but are not limited to: aliskiren, ditekiren, enalkiren, remikiren, terlakiren, ciprokiren and zankiren, pharmaceutically acceptable salts thereof, and mixtures thereof.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methylprednisolone, triamcinolone, fluocinolone, aldosterone, spironolactone, danazol (otherwise known as OPTINA), and others. In some embodiments, the therapeutic agent may comprise a steroid.

The terms “peroxisome proliferator-activated receptor gamma agent,” or “PPAR-y agent,” or “PPARG agent,” or “PPAR-gamma agent” refers to agents which directly or indirectly act upon the peroxisome proliferator-activated receptor. This agent may also influence PPAR-alpha, “PPARA” activity.

In some embodiments, the therapeutic agent may comprise a modulator of macrophage polarization. Illustrative modulators of macrophage polarization include peroxisome proliferator activated receptor gamma (PPAR-g) modulators, including, for example, agonists, partial agonists, antagonists or combined PPAR-gamma/alpha agonists. In some embodiments, the therapeutic agent may comprise a PPAR gamma modulator, including PPAR gamma modulators that are full agonists or partial agonists. In some embodiments, the PPAR gamma modulator is a member of the drug class of thiazolidinediones (TZDs, or glitazones). By way of non-limiting example, the PPAR gamma modulator may be one or more of rosiglitazone (AVANDIA), pioglitazone (ACTOS), troglitazone (REZULIN), netoglitazone, rivoglitazone, ciglitazone, rhodanine. In some embodiments, the PPAR gamma modulator is one or more of irbesartan and telmesartan. In some embodiments, the PPAR gamma modulator is a nonsteroidal anti-inflammatory drug (NSAID, such as, for example, ibuprofen) or an indole. Known inhibitors include the experimental agent GW-9662. Further examples of PPAR gamma modulators are described in WIPO Publication Nos. WO/1999/063983, WO/2001/000579, Nat Rev Immunol. 2011 Oct. 25; 11(11):750-61, or agents identified using the methods of WO/2002/068386, the contents of which are hereby incorporated by reference in their entireties.

In some embodiments, the PPAR gamma modulator is a “dual,” or “balanced,” or “pan” PPAR modulator. In some embodiments, the PPAR gamma modulator is a glitazar, which bind two or more PPAR isoforms, e.g., muraglitazar (Pargluva) and tesaglitazar (Galida) and aleglitazar.

In some embodiments, the therapeutic agent may comprise semapimod (CNI-1493) as described in Bianchi, et al. (March 1995). Molecular Medicine (Cambridge, Mass.) 1 (3): 254-266, the contents of which is hereby incorporated by reference in its entirety.

In some embodiments, the therapeutic agent may comprise a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7,294,753 the contents of which are hereby incorporated by reference in their entireties. In some embodiments, the MIF inhibitor is (S,R)-3-(4-hydroxyphenyl)-4,5-dihydro-5-isoxazole acetic acid methyl ester (ISO-1), isoxazoline, p425 (J. Biol. Chem., 287, 30653-30663), epoxyazadiradione, or vitamin E.

In some embodiments, the therapeutic agent may comprise a chemokine receptor 2 (CCR2) inhibitor as described in, for example, U.S. patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872, CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH351125, TAK779, Teijin, RS-504393, compound 2, compound 14, or compound 19 (Plos ONE 7(3): e32864).

In some embodiments, the therapeutic agent may comprise an agent that modulates autophagy, microautophagy, mitophagy or other forms of autophagy. In some embodiments, the therapeutic agent may comprise sirolimus, tacrolimis, rapamycin, everolimus, bafilomycin, chloroquine, hydroxychloroquine, spautin-1, metformin, perifosine, resveratrol, trichostatin, valproic acide, Z-VAD-FMK, or others known to those in the art. Without wishing to be bound by theory, agent that modulates autophagy, microautophagy, mitophagy or other forms of autophagy may alter the recycling of intra-cellular components, for example, but not limited to, cellular organelles, mitochondria, endoplasmic reticulum, lipid or others. Without further wishing to be bound by theory, this agent may or may not act through microtubule-associated protein 1A/1B-light chain 3 (LC3).

In some embodiments, the therapeutic agent may comprise an agent used to treat cancer, i.e., a cancer drug or anti-cancer agent. Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may comprise a taxane or derivative or analog thereof. The taxane may be a naturally derived compound, a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™)

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

According to certain aspects, the therapeutic agent may comprise a biologic drug, particularly an antibody. According to some aspects, the antibody is selected from the group consisting of cetuximab, anti-CD24 antibody, panitumumab and bevacizumab.

Therapeutic agents as used in the present disclosure may comprise peptides, proteins such as hormones, enzymes, antibodies, monoclonal antibodies, antibody fragments, monoclonal antibody fragments, and the like, nucleic acids such as aptamers, siRNA, DNA, RNA, antisense nucleic acids or the like, antisense nucleic acid analogs or the like, low-molecular weight compounds, or high-molecular-weight compounds, receptor agonists, receptor antagonists, partial receptor agonists, and partial receptor antagonists.

Additional representative therapeutic agents may include, but are not limited to, peptide drugs, protein drugs, desensitizing materials, antigens, factors, growth factors, anti-infective agents such as antibiotics, antimicrobial agents, antiviral, antibacterial, antiparasitic, antifungal substances and combination thereof, antiallergenics, steroids, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, anti-Alzheimer's agents, antihypertensive agents, beta-adrenergic blocking agents, alpha-adrenergic blocking agents, nutritional agents, and the benzophenanthridine alkaloids. The therapeutic agent can further be a substance capable of acting as a stimulant, a sedative, a hypnotic, an analgesic, an anticonvulsant, and the like.

Additional therapeutic agents may comprise CNS-active drugs, neuro-active drugs, inflammatory and anti-inflammatory drugs, renal and cardiovascular drugs, gastrointestinal drugs, anti-neoplastics, immunomodulators, immunosuppressants, hematopoietic agents, growth factors, anticoagulant, thrombolytic, antiplatelet agents, hormones, hormone-active agents, hormone antagonists, vitamins, ophthalmic agents, anabolic agents, antacids, anti-asthmatic agents, anti-cholesterolemic and anti-lipid agents, anti-convulsants, anti-diarrheals, anti-emetics, anti-manic agents, antimetabolite agents, anti-nauseants, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, laxatives, mineral supplements, mucolytic agents, neuromuscular drugs, peripheral vasodilators, psychotropics, stimulants, thyroid and anti-thyroid agents, tissue growth agents, uterine relaxants, vitamins, antigenic materials, and so on. Other classes of therapeutic agents include those cited in Goodman & Gilman's The Pharmacological Basis of Therapeutics (McGraw Hill) as well as therapeutic agents included in the Merck Index and The Physicians' Desk Reference (Thompson Healthcare).

Other therapeutic agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Further examples of therapeutic agents include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, anti spasmodics, antimalarials, antihistamines, antiproliferatives, anti-VEGF agents, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

Further representative therapeutic agents include but are not limited to analgesics such as acetaminophen, acetylsalicylic acid, and the like; anesthetics such as lidocaine, xylocaine, and the like; anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, paclitaxel, taxol, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like; tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like; peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam®, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The therapeutic agent can also be an immunomodulator, including, for example, cytokines, interleukins, interferon, colony stimulating factor, tumor necrosis factor, and the like; immunosuppressants such as rapamycin, tacrolimus, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcus neoformans, Histoplasma capsulatum, Candida albicans, Candida tropicalis, Nocardia asteroids, Rickettsia ricketsii, Rickettsia typhi, Mycoplasma pneumoniae, Chlamydia psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the therapeutic agent can comprise an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the therapeutic agent can be a combination of Rifampicin (Rifampin in U.S.) and Minocycline.

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, betal, beta2, beta3, skeletal growth factor, bone matrix derived growth factors, and bone derived growth factors and mixtures thereof.

Cytokines useful as therapeutic agents include, but are not limited to, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, and TNF-β. Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as therapeutic agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12,13, 15, 16, 17 and 18.

### Methods of Using Disclosed Vitreous Substitutes

In various aspects, the disclosed vitreous substitutes can be used to treat a clinical condition, disorder or disease of the eye, i.e., an ophthalmological disorder, in which the clinical condition, disorder, or disease is associated with undesirable levels of reactive oxygen species and/or an oxygen imbalance, e.g., a higher oxygen level than a healthy subject such as found in the eye following a vitrectomy procedure.

“Administering” the disclosed vitreous substitutes comprising an antioxidant of the present disclosure may be accomplished by any means known to the skilled artisan. Injection of liquid formulations into the eye is achieved via an injection needle having a suitable gauge, such as a relatively small gauge needle, including, but not limited to, 21 gauge, 25 gauge, 27 gauge, 28 gauge, 30 gauge, 31 gauge, or smaller. Solid implants can be administered via trocar, needle trocar, or other methods known in the art. See, e.g., U.S. Pat. Nos. 7,906,136; 5,869,079; 7,625,582. Surgical implantation into the eye is known in the art as described in U.S. Pat. Nos. 6,699,493; 6,726,918; 6,331,313; 5,824,072; 5,766,242; 5,443,505; 5,164,188; 4,997,652; 4,853,224.

Accordingly, the present disclosure pertains to methods of treating an ophthalmological disorder comprising administering a disclosed vitreous substitute to an eye in need thereof. In some aspects, the eye is an eye present in human subject. In other aspects, the eye is a present in a non-human subject.

The ophthalmological disorder can be acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, a cancer, and glaucoma. In certain instances, the ophthalmological disorder is wet age-related macular degeneration (wet AMD), a cancer, neovascularization, macular edema, or edema. In a further particular aspect, the ophthalmological disorder is wet age-related macular degeneration (wet AMD).

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

“Ocular region” or “ocular site” means any area of the ocular globe (eyeball), including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include, but are not limited to, the anterior chamber, the posterior chamber, the vitreous cavity, the choroid, the suprachoroidal space, the conjunctiva, the subconjunctival space, the episcieral space, the intracorneal space, the subretinal space, sub-Tenon's space, the epicorneal space, the sclera, the pars plana, surgically-induced avascular regions, the macula, and the retina.

“Ophthalmological disorder” can mean a disease, ailment or condition which affects or involves the eye or one of the parts or regions of the eye. Broadly speaking, the eye includes the eyeball, including the cornea, and other tissues and fluids which constitute the eyeball, the periocular muscles (such as the oblique and rectus muscles) and the portion of the optic nerve which is within or adjacent to the eyeball.

“Glaucoma” means primary, secondary and/or congenital glaucoma. Primary glaucoma can include open angle and closed angle glaucoma. Secondary glaucoma can occur as a complication of a variety of other conditions, such as injury, inflammation, pigment dispersion, vascular disease and diabetes. The increased pressure of glaucoma causes blindness because it damages the optic nerve where it enters the eye. Thus, in one nonlimiting embodiment, by lowering reactive oxygen species, STC-1, or MSCs which express increased amounts of STC-1, may be employed in the treatment of glaucoma and prevent or delay the onset of blindness.

“Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic retinopathy, uveitis, proliferative vitreoretinopathy (PVR), sympathetic ophthalmia, Vogt-Koyanagi-Harada (VKH) syndrome, histoplasmosis, and uveal diffusion.

“Injury” or “damage” in relation to an ocular condition are interchangeable and refer to the cellular and morphological manifestations and symptoms resulting from an inflammatory-mediated condition, such as, for example, inflammation, as well as tissue injuries caused by means other than inflammation, such as chemical injury, including chemical burns, as well as injuries caused by infections, including but not limited to, bacterial, viral, or fungal infections.

“Intraocular” means within or under an ocular tissue. An intraocular administration of a drug delivery system includes administration of the drug delivery system to a sub-tenon, subconjunctival, suprachoroidal, subretinal, intravitreal, anterior chamber, and the like location. An intraocular administration of a drug delivery system excludes administration of the drug delivery system to a topical, systemic, intramuscular, subcutaneous, intraperitoneal, and the like location.

“Macular degeneration” refers to any of a number of disorders and conditions in which the macula degenerates or loses functional activity. The degeneration or loss of functional activity can arise as a result of, for example, cell death, decreased cell proliferation, loss of normal biological function, or a combination of the foregoing. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and capillary endothelial cells. Age-related macular degeneration, or ARMD, is the major macular degeneration related condition, but a number of others are known including, but not limited to, Best macular dystrophy, Stargardt macular dystrophy, Sorsby fundus dystrophy, Mallatia Leventinese, Doyne honeycomb retinal dystrophy, and RPE pattern dystrophies. Age-related macular degeneration (AMD) is described as either “dry” or “wet.” The wet, exudative, neovascular form of AMD affects about 10-20% of those with AMD and is characterized by abnormal blood vessels growing under or through the retinal pigment epithelium (RPE), resulting in hemorrhage, exudation, scarring, or serous retinal detachment. Eighty to ninety percent of AMD patients have the dry form characterized by atrophy of the retinal pigment epithelium and loss of macular photoreceptors. Drusen may or may not be present in the macula. There may also be geographic atrophy of retinal pigment epithelium in the macula accounting for vision loss. At present there is no cure for any form of AMD, although some success in attenuation of wet AMD has been obtained with photodynamic and especially anti-VEGF therapy.

“Drusen” is debris-like material that accumulates with age below the RPE. Drusen is observed using a funduscopic eye examination. Normal eyes may have maculas free of drusen, yet drusen may be abundant in the retinal periphery. The presence of soft drusen in the macula, in the absence of any loss of macular vision, is considered an early stage of AMD. Drusen contains a variety of lipids, polysaccharides, and glycosaminoglycans along with several proteins, modified proteins or protein adducts. There is no generally accepted therapeutic method that addresses drusen formation and thereby manages the progressive nature of AMD.

“Ocular neovascularization” (ONV) is used herein to refer to choroidal neovascularization or retinal neovascularization, or both.

“Retinal neovascularization” (RNV) refers to the abnormal development, proliferation, and/or growth of retinal blood vessels, e.g., on the retinal surface.

“Subretinal neovascularization” (SRNVM) refers to the abnormal development, proliferation, and/or growth of blood vessels beneath the surface of the retina.

“Cornea” refers to the transparent structure forming the anterior part of the fibrous tunic of the eye. It consists of five layers, specifically: 1) anterior corneal epithelium, continuous with the conjunctiva; 2) anterior limiting layer (Bowman's layer); 3) substantia propria, or stromal layer; 4) posterior limiting layer (Descemet's membrane); and 5) endothelium of the anterior chamber or keratoderma.

“Retina” refers to the innermost layer of the ocular globe surrounding the vitreous body and continuous posteriorly with the optic nerve. The retina is composed of layers including the: 1) internal limiting membrane; 2) nerve fiber layer; 3) layer of ganglion cells; 4) inner plexiform layer; 5) inner nuclear layer; 6) outer plexiform layer; 7) outer nuclear layer; 8) external limiting membrane; and 9) a layer of rods and cones.

“Retinal degeneration” refers to any hereditary or acquired degeneration of the retina and/or retinal pigment epithelium. Non-limiting examples include retinitis pigmentosa, Best's Disease, RPE pattern dystrophies, and age-related macular degeneration.

In various aspects, a method of treating an ophthamological disorder may comprise treatment of various ocular diseases or conditions of the retina, including the following: maculopathies/retinal degeneration: macular degeneration, including age-related macular degeneration (ARMD), such as non-exudative age-related macular degeneration and exudative age-related macular degeneration; choroidal neovascularization; retinopathy, including diabetic retinopathy, acute and chronic macular neuroretinopathy, central serous chorioretinopathy; and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coats disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical diseases: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, ocular histoplasmosis syndrome (OHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigment epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigment epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e., front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Other diseases or disorders of the eye which may be treated in accordance with the present invention include, but are not limited to, ocular cicatricial pemphigoid (OCP), Stevens Johnson syndrome and cataracts.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e., the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic retinopathy; uveitis; ocular histoplasmosis; infections, such as fungal or viral-caused infections; macular degeneration, such as acute macular degeneration, non-exudative age-related macular degeneration and exudative age-related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial or venous occlusive disease, retinal detachment, uveitic retinal disease; sympathetic ophthalmia; Vogt-Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal ganglion cells or retinal nerve fibers (i.e., neuroprotection).

In some embodiments, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation, vitrectomy, corneal transplantation, forms of lamellar keratectomy (DSEK, etc.), and radial keratotomy.

In particular embodiments, the disclosed vitreous substitute may be used in the treatment of a retinal tear. In other embodiments, the disclosed vitreous substitute may be used in the treatment of proliferative retinopathy.

In a further aspect, the method is adjunctive therapy to a vitrectomy. That is, the present disclosure pertains to methods of treating an ophthalmological disorder comprising administering the disclosed vitreous substitutes to an eye following a vitrectomy.

From the foregoing, it can be seen that aspects herein are well adapted to attain all the ends and objects hereinabove set forth together with other advantages which are obvious and inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It can be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1. Poly(HEMA-Co-Bac)/PVA Hydrogel as a Vitreous Substitute

HEMA was crosslinked using BAC in a PVA solution. Compositions with varying percentages of HEMA and PVA (from 100% HEMA to 100% PVA by weight) and BAC (1-5% molar ratio to HEMA) in water/ethanol were synthesized via free radical polymerization with ammonium persulfate as catalyst and tetramethylethylenediamine as accelerator. The gels were homogenized using tissue grinders and reduced to liquid using 1,4-dithiothreitol (DTT) (10 times molar ratio to crosslinker BAC) under vigorous stirring and N2 bubbling. The reduced gels were adjusted to pH 4 and washed using dialysis tubes in distilled water (pH 4, N2 bubbled, 20 times the volume of gel) for 3 days to remove unreacted monomers. The dialyzed polymer solutions were precipitated in 10 times excess volume of methanol. The precipitates were lyophilized 24 hours. The freeze-dried polymers were reconstituted in Dulbecco's phosphate buffered saline at 37° C. and oxidized in a humidified chamber to reform hydrogels.

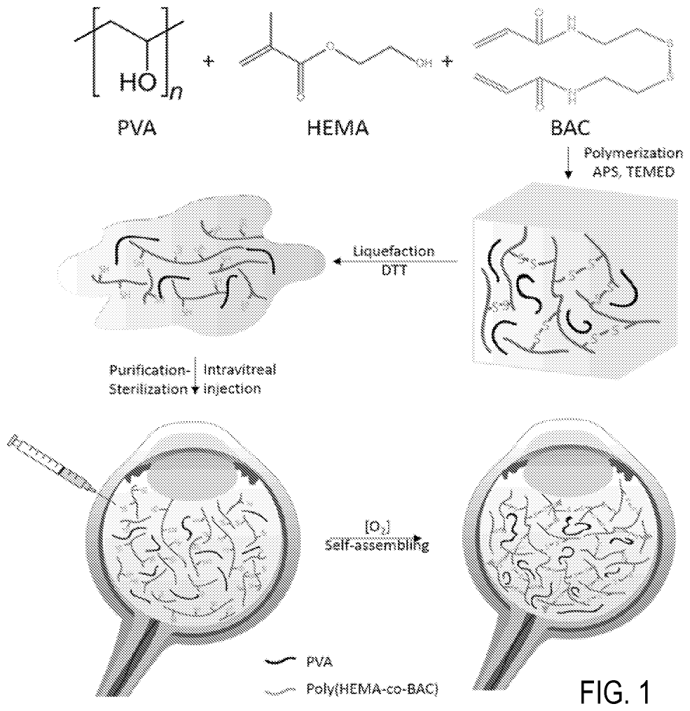

FIG. 1 shows the process of synthesizing an in-situ gelling poly(HEMA-co-BAC)/PVA hydrogel. After copolymerizing HEMA and BAC in the presence of PVA, the hydrogel was reduced to liquid using DTT. The disulfide cross-linking allows liquefaction of hydrogel for extensive purification and injection through a small-gauge needle. This semi-interpenetrating hydrogel resembles the microstructure of the natural vitreous humor, with the crosslinked poly(HEMA-co-BAC) serving as a rigid, collagen-like network of fibers and the hydrophilic PVA polymer chains, interspersed in the poly(HEMA-co-BAC) network, mimics the swelling hyaluronan molecules in the natural vitreous humor, providing the tamponade effect that inflates the posterior chamber of the eye wall. This injectable hydrogel is simple to use, which may seamlessly integrate into the current surgical vitrectomy procedure.

### Example 2. Preparation of Representative Disclosed Hydrogels

Hydrogels were prepared by free radical polymerization of 2-hydroxyethyl methacrylate (HEMA), poly(ethylene glycol) methacrylate (PEGMA), and poly(ethylene glycol) diacrylate (PEGDA) based on modifications of published protocols (Zellander A, et al. PloS one. 2014; 9:e96709). Briefly, HEMA:PEGMA:PEGDA copolymer hydrogels were polymerized in water. Ammonium persulfate and N,N,N′,N′-Tetramethylethylenediamine were used to initiate and catalyze the reaction. Ascorbic acid, an antioxidant with concentration 50 times higher in the eye than in blood (Holekamp N M. Am J Ophthalmol. 2010; 149:32-36), was encapsulated in gelatin-alginate particles as previously described (Comunian T A, et al. Food Res Int. 2013; 52:373-37). Briefly, Span 80 was added to an ascorbic acid solution to create an emulsion with corn oil. Gelatin and alginate were dissolved in water and slowly added to the water:oil emulsion with stirring for 30 min. The mixture was adjusted to pH 4.4 and stored at 4° C. for 12 h. The viscosity of the hydrogel was measured at different shear rates to determine its shear thinning capability using a Kinexus ultra+rheometer (Malvern Instruments Ltd, Worcestershire, UK). Ascorbic acid released from the encapsulating particles was determined using a Synergy HT multi-mode microplate reader (BioTek, Winooski, Vt.) at wavelength 265 nm.

### Example 3. Characterization of Representative Disclosed Hydrogels Comprising PEGMA

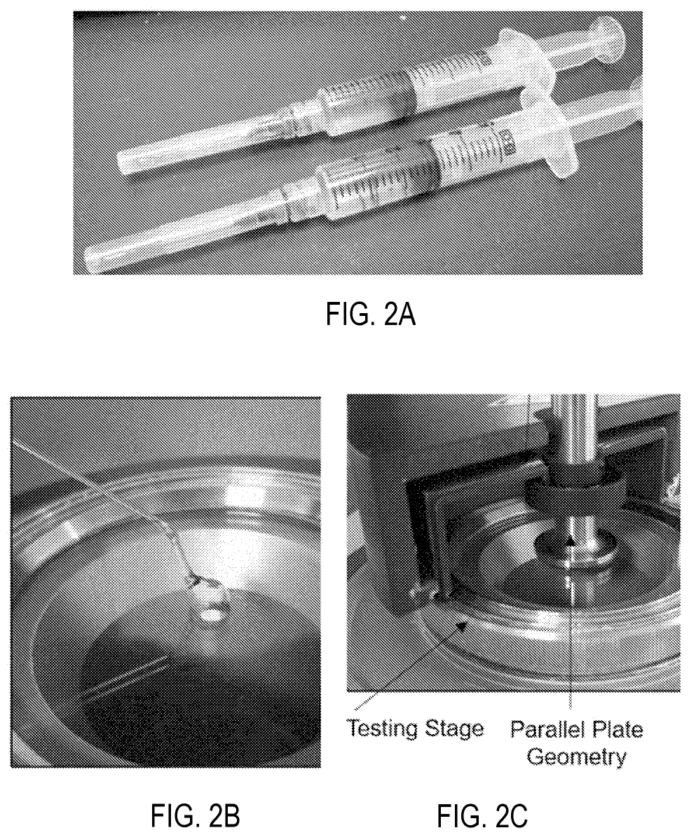

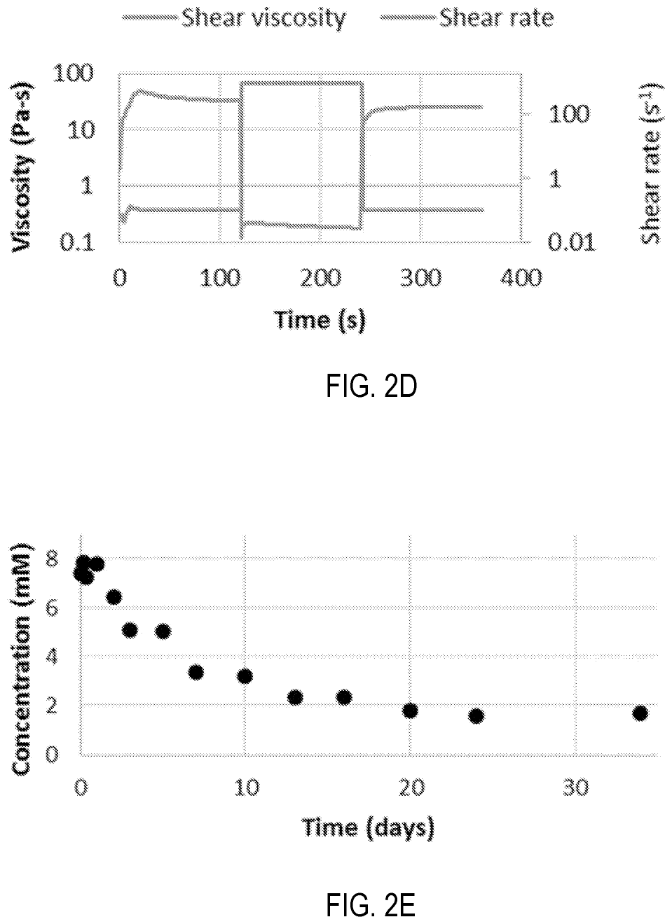

Preliminary formulations of HEMA:PEGMA:PEGDA were synthesized and produced clear, soft gels that shear thin and were easily injectable through a small gauge needle without compromising viscoelasticity, as evidenced by the storage (G′) and loss moduli (G″) before and after injection (FIGS. 2D-2E). The hydrogel had >90% transparency in visible light spectrum and diminished UV transmission. The encapsulation of ascorbic acid successfully prolonged its stability and release profile. The particles released ascorbic acid at 2 mM (normal concentration in the eye; Holekamp N M. Am J Ophthalmol. 2010; 149:32-36) for more than 30 days (FIG. 2E) and could be incorporated with the hydrogel during injection.

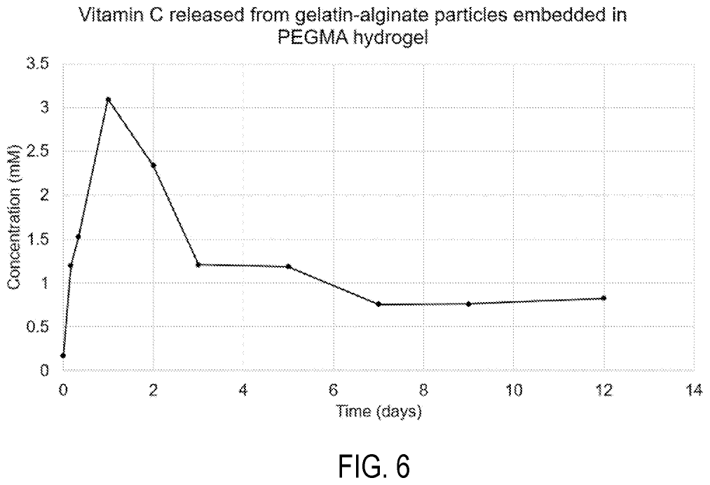

PEGMA hydrogel (20 ml, 5% v/v, MW 500) was synthesized then submerged in vitamin C solution (50 ml, 100 mM) for 12 h at room temperature. The hydrogel was placed in dialysis tubing and submerged in phosphate buffered saline (PBS, 70 ml). At predetermined times, the absorbance of PBS was measured at 265 nm to calculate the concentration of vitamin C release from PEGMA hydrogel. As shown in FIG. 5, the concentration of vitamin C released spiked to 50 mM within the first day, then rapidly diminished to near zero on subsequent days. In another experiment, the vitamin C-loaded gelatin-alginate particles were injected with the hydrogel through a 21G needle. The hydrogel/particles mixture was then submerged in PBS and the concentration of vitamin C in PBS was determined as aforementioned. The result showed a small spike in the release of vitamin C (compare to release from pure hydrogel above), followed by a period of sustained release of vitamin C as shown in FIG. 6.

### Example 4. Preparation and Characterization of Representative Disclosed Polyacrylamide Gels

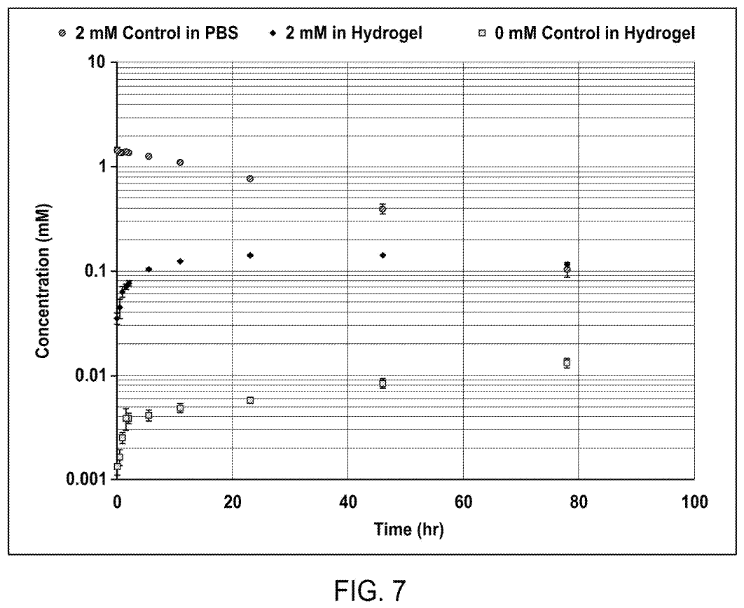

FIG. 7 shows the degradation profile of 2 mM sodium ascorbate solutions (n=3) and sodium ascorbate release profile from polyacrylamide hydrogels (n=3) at 37° C. with constant stirring. The 2 mM sodium ascorbate solutions in PBS, which was diluted 20× before measurement as previously described, show an exponential-like decay in concentration over time (note that the y-axis is plotted on a log scale). Additionally, its concentration at time 0 was 1.4 mM, not 2 mM as made, since there was a lag time between when the solutions were made and when the experiment started. This lag time (about 36 hours) was due to the delayed gelation time of polyacrylamide hydrogels. The polymer solutions with sodium ascorbate gelled within 18 hours. However, the polymer solutions without sodium ascorbate took twice as long to gel.

The gelled polyacrylamide hydrogels (1 ml) with or without sodium ascorbate were submerged in 10 ml of PBS. At predetermined times, 1 ml aliquots of the PBS solutions were obtained, and 1 ml fresh PBS was added to each sample to maintain sink condition (10× the volume of saturated solution, e.g. hydrogel). The 1 ml aliquots were measured without dilution, since the sodium ascorbate concentrations were already within the linear region of the standard curve.

The absorbance readings of the hydrogels without sodium ascorbate increase with time. Since there was no sodium ascorbate added to these hydrogels, the increase in absorbance could be due to the small pieces of polymer leached out from the hydrogel causing UV light interference. The hydrogels with sodium ascorbate likely have the same effect. The absorbance readings of hydrogels without sodium ascorbate can be subtracted from the ones with sodium ascorbate to obtain the true absorbance reading due to the varying concentrations of sodium ascorbate.

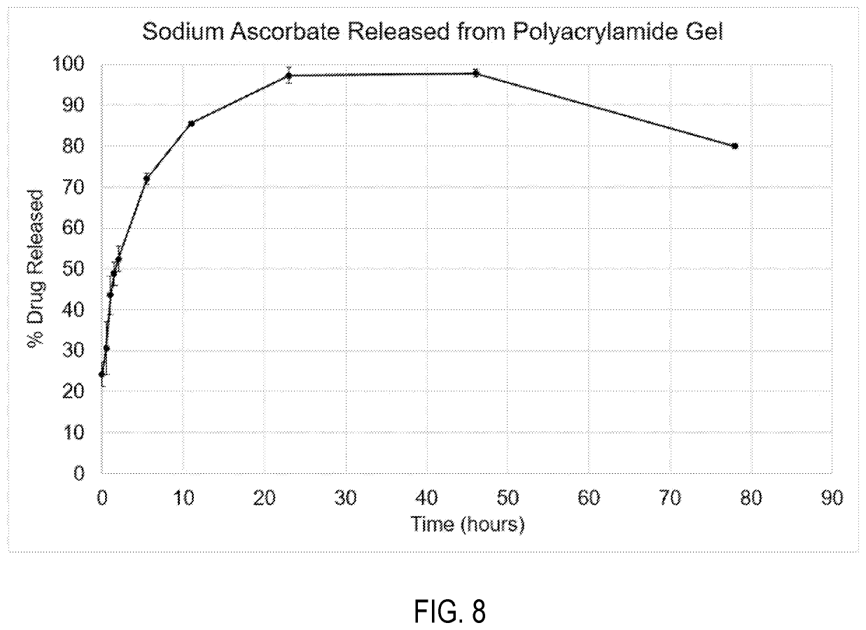

FIG. 8 shows the % sodium ascorbate released from polyacrylamide gel over 3 days, compared to the concentration of the 2 mM sodium ascorbate solutions at time 0 (which was 1.4 mM). Sodium ascorbate appeared to be fully released by the end of the first day. The % drug release on the third day decreases due to the degradation of sodium ascorbate.

### Example 5. Preparation and Characterization of Representative Disclosed Particles

Chitosan (Sigma-Aldrich, low molecular weight, 1 mg/ml) was dissolved in acetic acid solution (1% w/w, 500 ml) for 60 min at 500 rpm. Sodium tripolyphosphate (1.75 mg/ml, 500 ml) was added dropwise into the chitosan solution to form nanoparticles over 2 hours. The nanoparticles were collected by centrifugation at 4000 rpm for 15 min at 21° C. The particles were washed with deionized water and again centrifuged. Vitamin C (10% w/w, 10 ml, pH 5.5) was added to the particles and equilibrated for 18 hours on an orbital shaker. Sodium alginate (FMC BioPolymer, Protanal PH, 1 mg/ml, 10 ml, pH 5.5) was added to the vitamin C and chitosan particle solution and sonicated for 30 min. Chitosan (Sigma-Aldrich, low molecular weight, 1 mg/ml in 1% w/w acetic acid solution, 10 ml) or gelatin (bloom 175, 1 mg/ml, 10 ml) was added to the vitamin c-chitosan-alginate particles and sonicated for 30 min. The particles were collected by centrifugation at 4000 rpm for 15 min at 21° C. and freeze dried. Glutathione (1% w/w) can be incorporated with vitamin C into the particles. Concentration of vitamin C versus time was determined using methods as described elsewhere in the Examples. In some instances, the stability of vitamin C in the presence of glutathione was assessed.

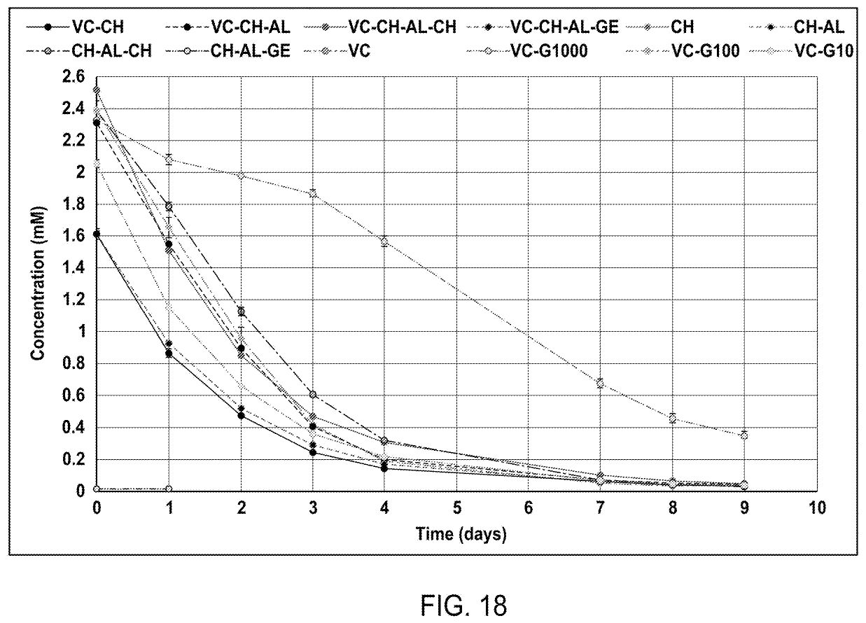

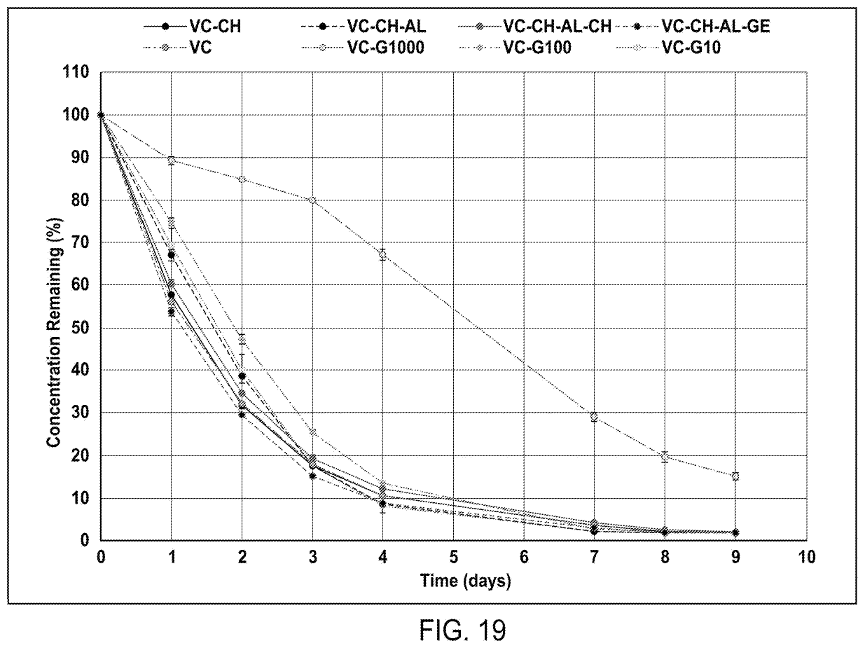

Date are presented in FIGS. 18-19 for release of antioxidant from the particles prepared as described above. Specifically, FIG. 18 shows representative data for release of ascorbic acid from representative disclosed particles comprising ascorbic acid loaded chitosan particles coated with alginate, chitosan, and/or gelatin as indicated. The legend in the figure uses the following abbreviations for detailing the composition of the particle: VC denotes vitamin C; CH denotes chitosan; AL denotes alginate; GE denotes gelatin; and “GXXX” denotes glutathione, with the concentration (μM) indicated by the number “XXX” as shown. The particles were prepared as described in the examples. FIG. 19 shows the data in FIG. 18, but with the vitamin C concentrations were normalized to the concentration at day 0. The data show improved maintenance of vitamin C concentrations in the presence of glutathione.

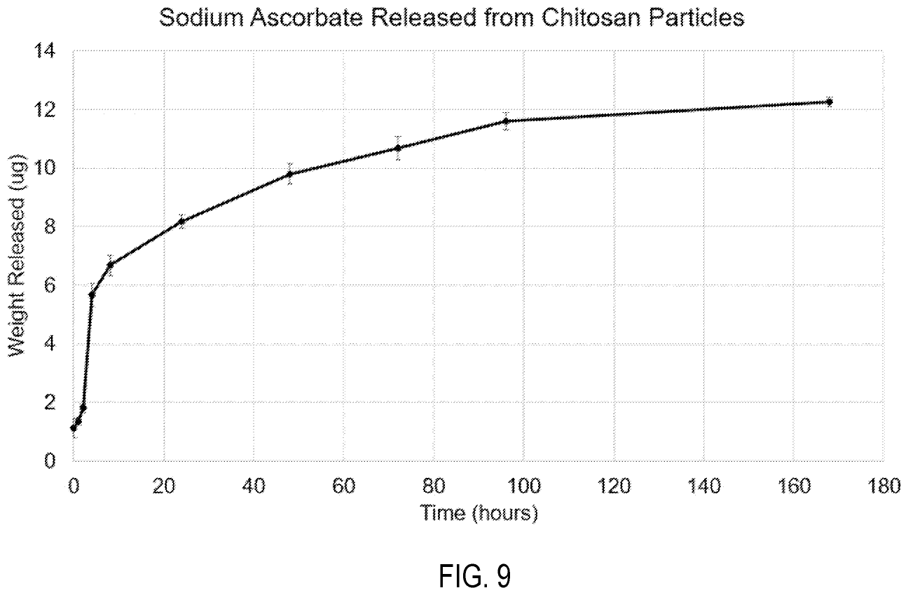

FIG. 9 shows additional data for sodium ascorbate release from chitosan particles. The study was done at room temperature with agitation (orbital shaker). The drug release (%) was not determined in this study, since sodium ascorbate was loaded during the chitosan particle synthesis. The subsequent washing steps after the formation of chitosan particles likely diminished the actual amount of sodium ascorbate loaded in the particles. Nonetheless, the release profile shows a more sustained released comparing to the release profile from polyacrylamide hydrogels, with the sodium ascorbate continuing to be released even after 7 days.

### Example 6. Prospective Characterization of Representative Disclosed Hydrogels

Refractive index can be determined using an Abbe refractometer, and light transmission can be evaluated in the UV and visible light ranges, with a target of over 90% light transmission in the visible light range. Representative hydrogel formulations demonstrate >90% transmission above 400 nm, and diminished UV transmission (FIG. 5), which would be desirable for protecting the retina if the lens, a UV light blocker, is removed for cataract surgery. Zeta potential and particle size of nanoparticles can be determined using light scattering and transmission electron microscopy.

Viscoelastic properties of the hydrogels can be characterized using a dynamic shear rheometer (Malvern Instruments Kinexus ultra+). After the linear viscoelastic region is determined, amplitude, frequency, and steady shear sweeps can be conducted. The biomechanical properties of the vitreous have previously been characterized and reviewed, and this data can be used to match the mechanical properties of a prepared disclosed hydrogel to those of the vitreous (Swindle-Reilly K E, Reilly M A, Ravi N. Current concepts in the design of hydrogels as vitreous substitutes. In Biomaterials and regenerative medicine in ophthalmology, 2nd edition. Chirila T V, Harkin D, eds. Ch 5. Woodhead Publishing Limited, 2016; and K. E. Swindle, P. D. Hamilton, N. Ravi, J. Biomed. Mater. Res. A. 2008, 87, 656-665). Representative data (FIG. 2D) demonstrate the ability to produce a gel with these properties.

In various aspects, disclosed hydrogels have properties similar to the vitreous humor: refractive index (1.336±0.002), moduli (G′ 10-20 Pa, G″ 1-10 Pa; ibid), and light transmittance (>90%). Particle size should be minimized (preferably <300 nm) to prevent visual impairment.

In vitro cytotoxicity of disclosed hydrogel formulations (with and without nanoparticles) can be assessed with lens epithelial cells (LEC) and human retinal pigment epithelial (ARPE-19) cells. A standard colorimetric 3-(4,5-dimethylthiazol-2-yl)-2,5-diphenyltetrazoliumbromide salt (MTT) assay can be used. Briefly, cells are seeded in 24-well plates at a density of 5×10⁴ cells/mL for 24 hours to achieve confluence. Cells can be incubated with gels for 24-48 hours. MTT reagent can be added to each well and Hoechst 33342 stain can be added to visualize cell nuclei. Plates can be read on a plate reader at 570 nm for MTT stain and 460-490 nm for nuclei stain, and cell viability can be calculated as a percentage of the untreated control. A standard live/dead viability assay may also be used to verify results from the MTT assay.

In various aspects, disclosed hydrogels have cell viability not significantly different from a negative control, as determined by t-tests (p<0.05). It is believed that the disclosed hydrogels are not associated with any remarkable cytotoxicity.

To evaluate the innocuity of the hydrogel vitreous substitutes (with and without nanoparticles) and their ability to mitigate oxidative stress in the lens, primary LECs can be cultured in transwells (Chandler H L, et al, Mol Vis, 2007 13:677-91). The use of transwells allow exposure of the LECs to the vitreous substitute without making direct contact, more closely mirroring the in vivo environment. The use of primary LECs can allow maintenance of key epithelial characteristics without induction of the transformative changes observed with immortalized cell lines (Wang-Su S T, et al, Invest Ophthalmol Vis Sci, 2003 44:4829-36). As a consequence, primary LECs have limited population doublings, and are most beneficial in the study of acute responses to treatment. To evaluate the longer-term effects of treatment, concurrent experiments using whole lenses can be performed. Whole lenses have an intact lens capsule and the lens fibers are retained; this can accurately model the effects of in vivo oxidative stressors (Kamiya T, Zigler J S, Exp Eye Res, 1996 63:425-31). In addition, whole lenses can be directly cultured on top of the substitutes, which is similar to what can be observed in vivo.

To evaluate the ability of the disclosed hydrogels comprising an antioxidant to prevent oxidation compared to silicone oil, cultured LECs and whole lenses can be exposed to environmental stimuli known to induce oxidative stress and contribute to cataract formation following vitrectomy (i.e. ultraviolet radiation, hydrogen peroxide, hyperoxide conditions). Stressed cells can be incubated in the presence of the test materials and cellular viability can be evaluated using an MTT assay. Production of reactive oxygen species can be determined using a standard dichlorofluorescein (DCF) assay. Additional outcome measures to quantify the anti-cataractogenic properties of the vitreous substitutes include determining glutathione (GSH) concentration (Harding J J, Biochem J, 117:957-60, 1979), glutathione reductase (GR) activity (Linetsky M D, et al, Biochim Biophys Acta, 1724:181-93, 2005), protein-bound GSH, catalase activity (Beers R F, Sizer I W, J Biol Chem, 195:133-40, 1952), Na⁺-K⁺-ATPase activity (Akagawa K, Tsukada Y, J Neurochem, 32:269-71, 1979), and ascorbate concentration (Okamura M, Clin Chim Acta, 103:259-68, 1980).

In various aspects, the disclosed hydrogels show a significant reduction of reactive oxygen species and significant differences in assay measurements for anti-cataractogenic properties (p<0.05) compared to controls (untreated and silicone oil) as determined by ANOVA. antioxidant activity can be quantified, and ascorbate concentration can be directly measured (ibid).

The safety and efficacy of the vitreous substitutes can be evaluated in a rabbit vitrectomy model. All studies are conducted using an IACUC-approved protocol and abide by The Association for Research in Vision and Ophthalmology (ARVO) Statement for the Use of Animals in Ophthalmic and Vision Research. The vitreous substitutes can be evaluated using Dutch belted rabbits, the standard animal model for evaluation of vitreous substitutes (Del Amo E M, Urtti A, Exp Eye Res, 137:111-24, 2015). After purchase, rabbits acclimate to surroundings at a University Laboratory Animal Resources facility for 5-7 days. Rabbits can be divided into 3 treatment groups to evaluate the hypotheses that a gel formulation would prevent damage compared to silicone oil, and that the incorporation of the antioxidant prevents oxidative damage to the lens and retina. Following pars plana vitrectomy, the vitreous in one eye can be replaced with hydrogel vitreous substitute (n=6), hydrogel with antioxidant-loaded nanoparticles (n=6), or silicone oil positive control (n=6; e.g., for methods see Del Amo E M, Urtti A, Exp Eye Res, 137:111-24, 2015; and K. E. Swindle-Reilly, M. Shah, P. D. Hamilton, T. A. Eskin, S. Kaushal, N. Ravi, Invest. Ophthalmol. Vis. Sci. 2009, 50, 4840-4846). The fellow eye in each rabbit serves as an untreated control. Equal numbers of male and female rabbits can be evaluated in each test group to account for biological variability. Rabbits can be monitored as detailed below for 60 days after vitrectomy, then humanely euthanized. Globes can be harvested for histopathological evaluation.

Prior to vitrectomy, all rabbits can undergo a complete dilated ophthalmic examination including TOP measurement (Tonovet), slit lamp biomicroscopy (Kowa SL-15), and indirect ophthalmoscopy (Heine Omega 500). Additionally, electroretinogram (ERG), refraction by retinoscopy (Welch Allyn), and OCT (Envisu) can be performed. Anterior segment and fundus photographs can be taken. Postoperatively, rabbits can receive a complete ophthalmic examination as above on the first postoperative day, at one week, and then weekly until the conclusion of the study. Any clinically evident anterior segment changes identified via slit-lamp biomicroscopy (e.g. conjunctival hyperemia, aqueous flare, iridal hyperemia, loss of corneal transparency) can be objectively quantified with a modified Hackett-McDonald scoring system (Hackett R B, McDonald T O, Dermatotoxicology, 1996). Posterior segment changes including vitreous haze or retinal changes can be quantified using the Nussenblatt scoring system for posterior uveitis (Sen H N, et al, Ophthalmology, 118(4):768-71, 2011). ERG, refraction by retinoscopy, and OCT can be repeated at the mid-point (1 month post-operatively), and at the end of the study.

After anesthetizing the rabbits, the eyelids of one eye can be swabbed with betadine 3x. Using a surgical microscope (Zeiss), 23 gauge trochars can be placed 2.0 mm behind the limbus at the 2- and 10-o'clock positions. Vitrectomy can then be done under direct visualization through a contact lens on the cornea. Air-fluid exchange can be done using a back-flush brush. At that time the experimental vitreous substitutes or silicone oil can be injected into the eye. At the end of surgery, the trochars can be removed. No sutures are required to secure the sclerotomies because the wounds are self-sealing. This procedure mimics that performed in human patients. Post-operative treatment protocols include analgesics for pain control as well as topical medications to prevent surgical related inflammation and post-operative infections.

Fresh tissue can be harvested from a subset of whole eyes to quantify antioxidant markers. Following dissection, whole lenses can be weighed and frozen until further analysis. All lenses can be homogenized in sterile saline and centrifuged. Clear supernatant can be used for all subsequent experiments. As described above, antioxidant activity can be quantified (e.g. GSH concentration, CAT activity). The concentration of ascorbate in the lens, aqueous humor, and fluid within the vitreal chamber can be determined (Okamura M, Clin Chim Acta, 103:259-68, 1980).

Hematoxylin and eosin (H&E) and immunofluorescence can be conducted on subsets of tissue. Tissue samples can be immediately fixed in 4% paraformaldehyde. After gross examination, both the anterior and posterior segment cups can be dissected, and a subset can be embedded in paraffin for histology and immunohistochemistry to investigate morphology and retinal layer thickness while the remaining tissue can be frozen for additional analysis. Three consecutive sections can be obtained from the posterior and anterior segments of each eye and stained with H&E by a veterinary histologist. Lens and retinal pathology can be evaluated for cataract and oxidative damage using clinical scoring, and a pathologist will review sections. Retinal sections can be evaluated for GFAP and CD68 to evaluate microglia activation, and cell death for toxicity. ERGs, refraction, and gross morphology will also be used to monitor retinal health. When RPE cells respond to excessive oxidative stress, they yield TUNEL-positive cells (Sen H N, et al, Ophthalmology, 118(4):768-71, 2011). Additional analyses can include staining the retina and lens for markers to evaluate oxidative stress (e.g. TNF-α, IL-1-β, TUNEL; for method, see Kim B, et al, Sci Rep, 7:14336, 2017).

In various aspects, the disclosed hydrogels show in vivo normal ERG, histology, and IOP; minimal inflammation and cytotoxicity; and less oxidative damage to the lens and retina compared to the silicone oil control. Quantifiable measures to evaluate for statistical significance compared to silicone oil and untreated control can include ERG changes, TOP, microglia, retinal layer thickness, histopathology, refraction, cataract grading, and slit lamp observation scores.

A shear-thinning hydrogel embedded with antioxidant releasing particles was created as a novel vitreous substitute that can replace both the physical and chemical functions of the natural vitreous humor. The maintenance of the natural oxygen gradient by this vitreous substitute has the potential to prevent post-vitrectomy cataract formation, significantly reducing the cost of additional treatments for patients and health care providers.

### Example 7. Disclosed Hydrogels as Vitreous Substitutes for Antioxidant Release

Experimental Section—Materials. Poly(ethylene glycol) methacrylate (PEGMA, average molecular weight (MW) 360), poly(ethylene glycol) diacrylate (PEGDA, average MW 250, 575, and 700), N,N,N′,N′-Tetramethylethylenediamine (TEMED), ammonium persulfate (APS), and Dulbecco's phosphate-buffered saline (DPBS) were purchased from Sigma-Aldrich (St. Louis, Mo., USA) and used without further purification. 2-Hydroxyethyl methacrylate (HEMA) was purchased from Monomer Polymer & Dajac Labs (Ambler, P A, USA). Dialysis tubing with molecular weight cut off (MWCO) of 6-8 kDa and 12-14 kDa, Dulbecco's Modified Eagle's/Nutrient Mixture F-12 Ham's Medium (DME/F-12), Dulbecco's Modified Eagle's Medium (DMEM), DMEM without phenol red, fetal calf serum (FCS), Penicillin-Streptomycin (Pen Strep), trypsin, lysozyme, and hydrogen peroxide were purchased from Thermo Fisher Scientific (Waltham, Mass., USA) and used as received. RPE (ARPE-19 ATCC CRL-2302) were purchased from American Type Culture Collection (Manassas, Va., USA). LEC cells are an immortalized human lens epithelial cell line, i.e., immortalized SRA 01/04 human LEC. The cell line was produced by transfection of human lens epithelial cells with plasmid vector DNA containing a large T antigen of SV40.33 (N. Ibaraki, et al., Exp Eye Res. 1998, 67, 577-585). CellTiter-Glo Luminescent Cell Viability Assay was purchased from Promega (Madison, Wis., USA). Dichlorofluorescein (2,7-Dichlorodihydrofluorescein diacetate, DCF) was purchased from Cayman Chemical (Ann Arbor, Mich., USA).

Preparation of disclosed hydrogels. Multiple copolymers of HEMA, PEGDA, and PEGMA were synthesized in deionized water (pH 7.4) and screened based on transparency and mechanical properties (Table 1). The hydrogels were formed by free radical polymerization as previously published with modifications.^{[31]} Briefly, HEMA, PEGMA, and PEGDA monomers were dissolved in deionized water and extensively purged with nitrogen gas to remove oxygen molecules that might terminate the reaction prematurely. APS aqueous solution (10% w/v) and TEMED were added as free radical initiator and accelerator at 1:200 and 1:800 v/v, respectively. [32] The solutions were allowed to polymerize for 12 hours. The hydrogels were purified against deionized water for 7 days in dialysis tubing (12-14 kDa MWCO) to remove unreacted monomers and low molecular weight polymer chains. Two optimized formulations were created, namely PEGDA and PEGDA-co-PEGMA hydrogels (Table 1).

TABLE 1
Example 6 - Hydrogel formulations.

| Formulation | PEGDA | PEGDA | PEGMA | HEMA | | | |
|---|---|---|---|---|---|---|---|
| Name | MW | wt % | wt % | wt % | Transparent? | Gel? | Consistency |
| Formulation 1 | 575 | 3% | 3% | 0% | Yes | Yes | Hard |
| Formulation 2 | 575 | 1.2% | 4.8% | 0% | Yes | Yes | Hard |
| Formulation 3 | 575 | 0.3% | 5.7% | 0% | Yes | Yes | Soft |
| Formulation 4 | 575 | 1.2% | 2.4% | 2.4% | No | Yes | Hard |
| Formulation 5 | 575 | 1.2% | 2.1% | 0.3% | Yes | Yes | Hard |
| Formulation 6 | 575 | 1.2% | 0.3% | 2.1% | No | Yes | Hard |
| Formulation 7 | 250 | 1% | 0% | 0% | No | Yes | Hard |
| Formulation 8 | 250 | 0.75% | 0% | 0% | No | No | Liquid |
| | 575 | 0.75% | | | | | |
| Formulation 9 | 250 | 0.75% | 0.75% | 0% | No | No | Liquid |
| Formulation 10 | 575 | 1% | 0% | 1% | No | Yes | Hard |
| Formulation 11 | 250 | 1% | 1% | 1% | No | No | Liquid |
| Formulation 12 | 700 | 1% | 1% | 1% | No | No | Liquid |
| Formulation 13 | 575 | 3% | 0% | 0% | Yes | Yes | Hard |
| Formulation 14 | 575 | 1% | 0% | 0% | Yes | No | Liquid |
| Formulation 15 | 250 | 1.5% | 0% | 0% | No | Yes | Hard |
| Formulation 16 | 575 | 1.5% | 0% | 0% | Yes | No | Liquid |
| Formulation 17 | 700 | 1.5% | 0% | 0% | Yes | No | Liquid |
| Formulation 18 | N/A | 0% | 6% | 0% | Yes | Yes | Soft |
| Formulation 19 | 575 | 2% | 0% | 0% | Yes | Yes | Soft |
| (PEGDA) | | | | | | | |
| Formulation 20 | 575 | 1.5% | 1.5% | 0% | Yes | Yes | Soft |
| (PEGDA-co- | | | | | | | |
| PEGMA) | | | | | | | |

Rheology Determination. Prior to measurement, all hydrogel samples were immersed in DPBS for 7 days to reach equilibrium swelling. The samples were syringed onto the quartz testing stage of a Kinexus ultra+rheometer (Malvern Instruments Ltd, Worcestershire, UK). A 20-mm parallel plate geometry was lowered onto the hydrogel sample to a working gap of 1 mm, which was determined to provide good contact between the geometry and the hydrogel without damaging the sample (zero normal force). The testing stage was set to 37° C. and a humidifying chamber filled with DPBS was attached around the geometry and testing stage to simulate in vivo conditions and prevent sample dehydration (FIGS. 2A-2C). Amplitude sweep tests were conducted at a frequency of 0.1 Hz and amplitudes ranging from 0.1 to 1000%. Frequency sweep tests with strain amplitude of 1% (found to be within the linear viscoelastic region) were conducted with frequency ranges from 0.01 to 1 Hz to determine the storage modulus (G′) and loss modulus (G″) of the hydrogels. Shear viscosity was evaluated by increasing the shear rate from 0.01 to 1000 s⁻¹. Alternating oscillatory step strains were applied to the hydrogels at a fixed frequency of 0.1 Hz and strains of 10%, 700%, and 1000% with 100 s for each strain interval (H. Wang, et al., Adv. Sci. 2018, 5, 1800711).

Hydrogel Characterization. The equilibrium water content of each hydrogel formulation was determined by drying known amounts of water-swollen hydrogels in a 60° C. oven until no change in weight was detected. The refractive indices of the hydrogels were determined using a refractometer (Sper Scientific, Scottsdale, Ariz.). The transmittance of the hydrogel was measured using a Varian Cary 50 UV-Visible Spectrophotometer (Agilent Technologies, Santa Clara, Calif., USA) at wavelengths ranging from 230 to 900 nm. DPBS was used as a blank. Fourier-transform infrared spectra (FTIR) of the PEGDA and PEGDA-co-PEGMA hydrogels were collected using a Thermo Nicolet Nexus 870 FTIR spectrometer (Thermo Fisher Scientific, Waltham, Mass., USA).

Hydrogel Stability. The hydrogels were incubated with DPBS, lysozyme (10,000 U mL⁻¹), or trypsin (0.25%) at 37° C. for up to 4 weeks (S. Santhanam, et al., Acta Biomater. 2016, 43, 327-337). DPBS, lysozyme, or trypsin (1 mL each) was added to PEGDA or PEGDA-co-PEGMA hydrogels (0.5 g). At predetermined times (0, 1, 4, 7, 14, 21, and 28 days), the hydrogels were lyophilized and weighed. The weight stability of the hydrogel samples was determined by the given formula:

$$Weight Stability = \frac{{W}_{t}}{{W}_{0}} * 100$$

Where W₀ is the initial weight of the wet hydrogel at time 0 and Wt is the weight of the gel at time t (days).

Vitamin C Loading, Stability, and Release. Hydrogels were placed in low molecular weight cut-off dialysis tubing (MWCO 6-8 kDa) and immersed in vitamin C solution (2.2 mM, prepared fresh and changed daily) for 72 hours. The concentration of vitamin C in the human vitreous is 2 mM (N. M. Holekamp, Am J Ophthalmol. 2010, 149, 32-36). A vitamin C concentration of 2.2 mM was chosen as the loading concentration to account for the rapid degradation of vitamin C. To determine the stability of vitamin C in hydrogels, the vitamin C loaded hydrogels were kept at 37° C. At predetermined times (0 and 30 minutes, 1, 2, 4, 8, and 12 hours, 1, 2, 3, 4, and 7 days), the vitamin C remaining in the hydrogel was determined using a Synergy HT multi-mode microplate reader (BioTek, Winooski, Vt.) at wavelength 265 nm, compared against standard solutions with known concentrations with blank hydrogels as the background reading. To determine vitamin C release, the hydrogels were loaded with vitamin C solution (1% w/v) as aforementioned. A concentration of 1% w/v, or 5.7 mM, was chosen for the release study because lower loading concentrations resulted in lower concentrations of released vitamin C that were too low to be reliably detected. The vitamin C loaded hydrogels (4 mL for each sample) were placed in dialysis tubing (MWCO 6-8 kDa) and submerged in DPBS (100 mL). At predetermined times as described above, DPBS solution (1 mL) was withdrawn to determine the concentration of vitamin C released, and fresh DPBS (1 mL) was added to maintain sink condition.

Cell Viability and ROS Activity Assays. ARPE-19 and LEC were seeded in 96-well plates at 1×10⁴ cells per well in DMEM/F-12 and DMEM, respectively, supplemented with 10% FCS and 1% Pen Strep for 24 h at 37° C. in 5% CO₂ humidified atmosphere. The hydrogels were submerged in 70% ethanol for 1 hour to sterilize, rinsed with deionized water 3 times for 1 hour each to remove the residual ethanol, and mixed well with serum-free and phenol red-free DMEM at a hydrogel concentration of 10% w/v (J. Chang, et al., J Mater Chem B. 2015, 3, 1097-1105; Y. Tao, et al., Acta Biomater. 2013, 9, 5022-5030; M. Annaka, et al., Biomacromolecules. 2011, 12, 4011-4021; and S. Lamponi, et al., J. Biomater. Sci. Polym. Ed. 2012, 23, 555-575). The culture medium in each well was removed and medium (100 μL) with/without hydrogel and with/without vitamin C (2.2 mM) was added to each well and incubated for 24 hours. Hydrogen peroxide (10 200 μM final concentration) was added to half of the wells, and DPBS (10 μL) was added to the remaining wells as a control (A. Heckelen, et al., Acta Ophthalmol Scand. 2004, 82, 564-568; and H. S. Lee, et al., Invest. Ophthalmol. Vis. Sci. 2017, 58, 1196-1207). The well plates were incubated for 30 minutes. CellTiter-Glo luminescent cell viability assay was conducted according to the manufacturer's protocol. Briefly, the well plates were equilibrated to room temperature for 30 minutes. CellTiter-Glo Reagent (100 μL) was added to each well, and the contents were mixed for 10 minutes using an orbital shaker. The well plates were incubated at room temperature for 10 minutes before the luminescent signal was measured using the Synergy HT multi-mode microplate reader. ROS activity was detected using DCF. Briefly, DCF (100 20 μM final concentration) was added to each well, and the contents were incubated at room temperature for 30 minutes (Y. Ou, et al., Chem Biol Interact. 2009, 179, 103-109). The fluorescence signal was measured with excitation and emission wavelengths of 485 and 525 nm, respectively, using a TECAN M200 Plate Reader (Männedorf, Switzerland).

Intravitreal Hydrogel Injection. Porcine globes from six-month old pigs (Sioux-Preme Packing Co., Sioux City, Iowa) were shipped overnight in saline solution packed in ice. Extraocular tissues were removed from the eyes. An orifice was made through the lamina cribrosa using a 15G blunt cannula, through which the vitreous was removed. The hydrogels (4 mL) were injected into the vitreal chamber using a 22- or 30-gauge hypodermic needle. The ocular globe was transected to assess the appearance of hydrogels inside the vitreal chamber.

Statistical Analysis. Data are expressed as mean±standard error (SE). Statistical analyses were implemented with Minitab software (version 18.1; Minitab, Inc., State College, Pa.). One-way ANOVA, with post-hoc pairwise comparison using Tukey test, was used to analyze the rheological data, the hydrogel stability data, and the cell viability and ROS activity of the ARPE-19 and LEC. The null hypotheses stated that there is no difference between the groups for each test. An alpha value of 0.05 was used for statistical significance.

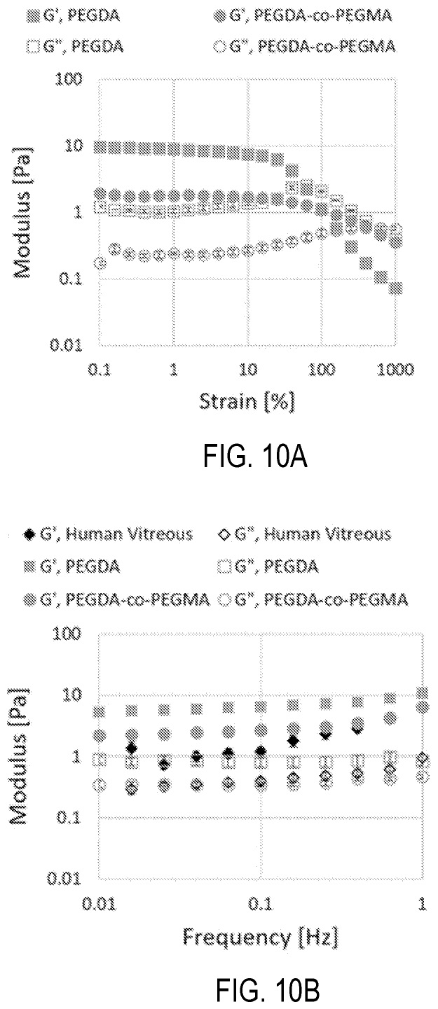

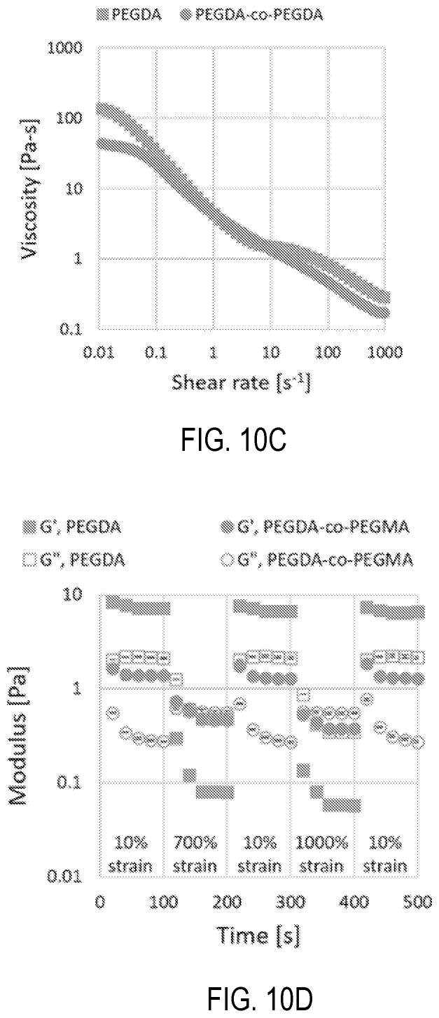

Results—Disclosed Hydrogels of Example 6. Rheological experiments showed viscoelastic properties of the PEGDA and PEGDA-co-PEGMA hydrogels similar to the native tissue (N. K. Tram, K. E. Swindle-Reilly, Front. Bioeng. Biotechnol. 2018, 6; and A. Schulz, et al., Transl Vis Sci Technol. 2019, 8, 56). The linear viscoelastic region for both hydrogels was determined to be below 10% strain (FIG. 10A). The storage modulus (G′) and loss modulus (G″) represent the elastic and viscous properties of a viscoelastic material, respectively. While both moduli decreased above 10% strain, the loss modulus became larger than the storage modulus, suggesting that the hydrogels were becoming more liquid-like. Therefore, a strain of 1% was used for the subsequent frequency sweep experiments. The moduli of the PEGDA hydrogel were statistically larger than those of the PEGDA-co-PEGMA hydrogel and human vitreous humor (G′_{PEGDA}=7.02±0.33 Pa>G′_{PEGDA-co-PEGMA}=3.16±0.22 Pa≈G′_{human vitreous}=2.368±0.17 Pa, p<0.0001; G″_{PEGDA}=0.859±0.038 Pa>G″_{PEGDA-co-PEGMA}=0.378±0.011 Pa≈G″_{human vitreous}=0.482±0.024 Pa, p<0.0001). The storage modulus (G′) and loss modulus (G″) of both hydrogels were in the same order of magnitude as the natural human vitreous (FIG. 10B). The storage modulus of the human vitreous ranges from 1 Pa to 7 Pa, whereas its loss modulus ranges from 0.3 Pa to 1 Pa (ibid). The storage and loss moduli of the PEGDA hydrogel were statistically larger than those of the natural human vitreous with the storage modulus ranging from 5 to 11 Pa and the loss modulus ranging around 0.9 Pa. The storage and loss moduli of PEGDA-co-PEGMA hydrogel were not statistically different than the reported properties of human vitreous, with the storage modulus ranging from 2 to 7 Pa and the loss modulus ranging around 0.4 Pa. Both hydrogels became less viscous as the shear rate increases, demonstrating shear thinning behavior, which is favorable for injection (FIG. 10C). Alternating oscillatory step strain experiments further showed that, after undergoing high strains that caused shear thinning of the hydrogels (G″>G′), both hydrogels quickly recovered their gel-like behavior at a lower strain (FIG. 10D).

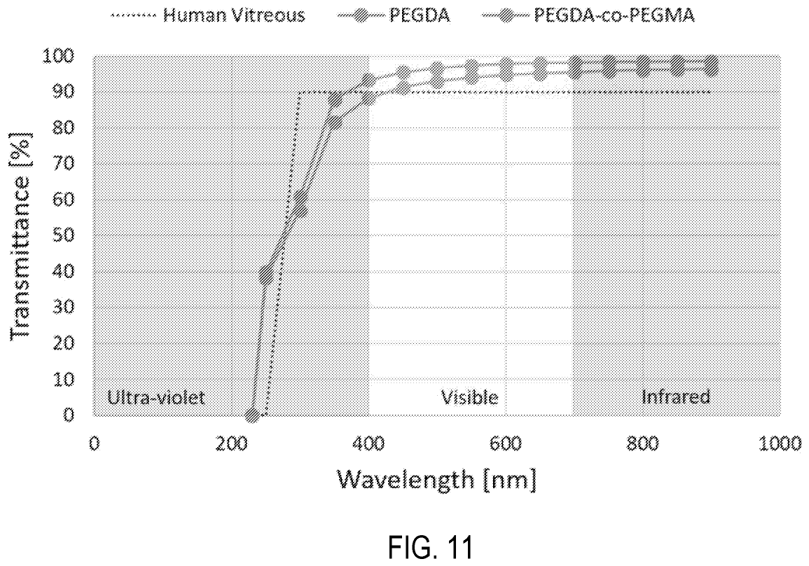

The hydrogels had acceptable transparency (above 90%) within the visible wavelengths (FIG. 11). The PEGDA-co-PEGMA hydrogel was more transparent than the PEGDA hydrogel, but both hydrogels had optical properties similar to the natural human vitreous (E. A. Boettner, J. R. Wolter, Invest Ophthalmol Vis Sci. 1962, 1, 776-783). The transmittance of the hydrogels rapidly dropped in the ultraviolet range to zero at 230 nm. Each hydrogel formulation also has a similar refractive index as the human vitreous, which is 1.3349 (B. P. Gloor, The CV Mosby Co., St. Louis. 1987, 246-267). The refractive index of the PEGDA hydrogel was 1.3350±0.0002, and the refractive index of the PEGDA-co-PEGMA hydrogel was 1.3359±0.0002. These excellent optical properties are likely due to the high water contents of the hydrogels. The equilibrium water contents of PEGDA and PEGDA-co-PEGMA hydrogels were 97.53±0.06% and 96.91±0.01%, respectively.

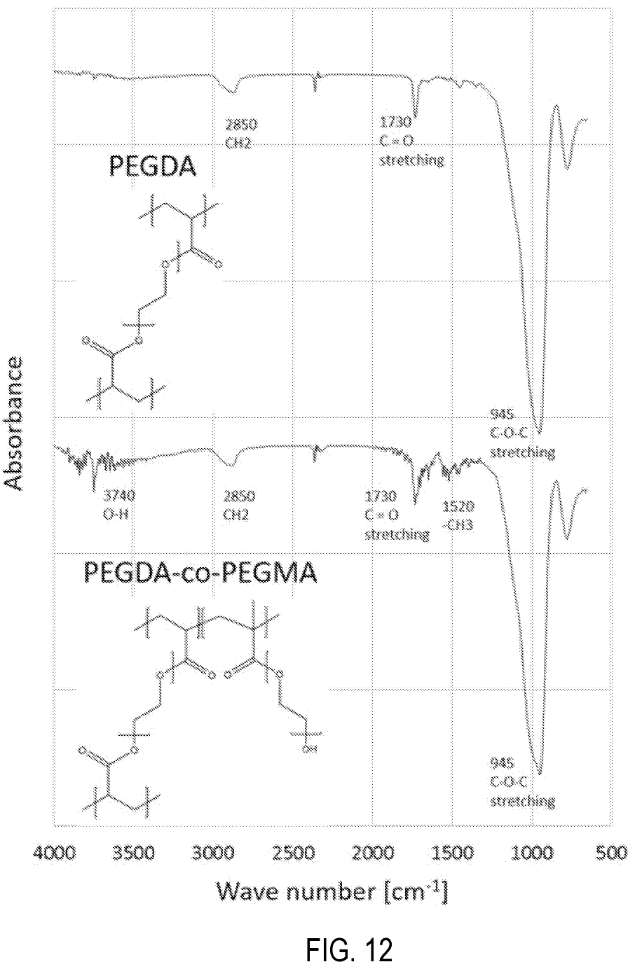

FTIR showed the successful synthesis of the PEGDA and PEGDA-co-PEGMA hydrogels (FIG. 12). The methylene (—CH2-), carbonyl (C═O), and ether (C—O—C) groups were found in both hydrogel spectra at 2850, 1730, and 945 cm⁻¹, respectively. The PEGDA-co-PEGMA hydrogel spectra showed the existence of the alcohol (—OH) and methyl (—CH3) groups at 3740 and 1520 cm⁻¹, respectively. These peaks did not appear in the PEGDA spectra, confirming that the appropriate hydrogels were synthesized.

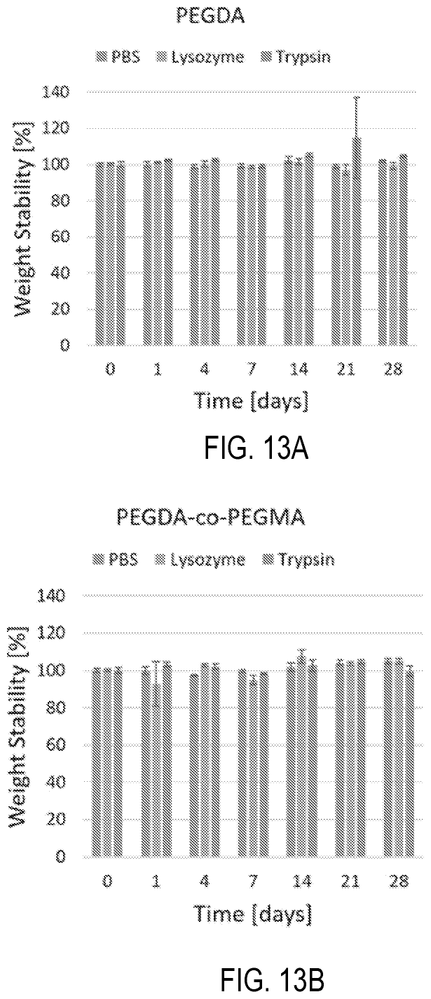

The hydrogels were found to be stable after incubation with enzymatic solutions (FIGS. 13A-13B). The hydrogel weight did not statistically change in DPBS, lysozyme, or trypsin solutions for at least 28 days at 37° C. for both hydrogels (p >0.05).

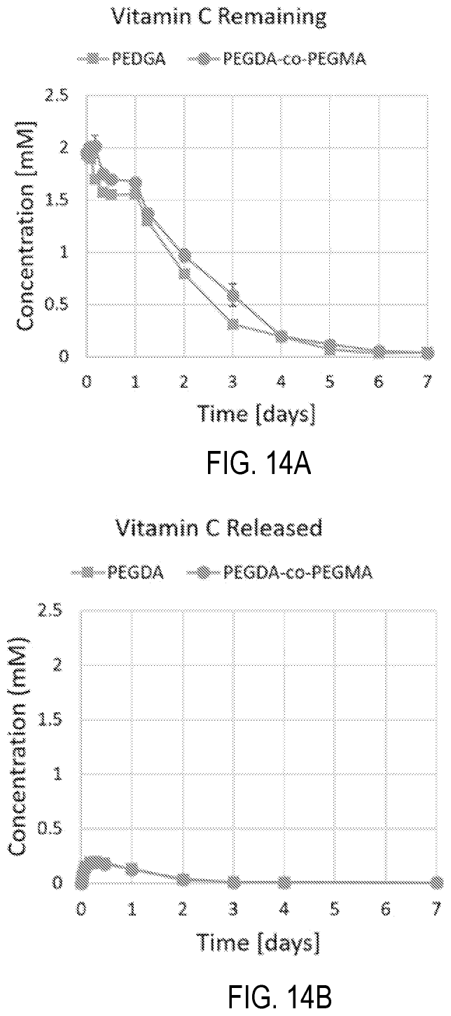

The hydrogels loaded with vitamin C showed quick degradation of vitamin C (FIG. 14A) in the vitamin C stability experiment. The first rapid drop of vitamin C occurred within the first 8 hours, from 2 mM to around 1.6 mM. Thereafter, the vitamin C concentration inside the hydrogels decreased to 0.03 mM after 7 days. Rapid release of vitamin C also occurred during the first 8 hours (FIG. 14B) in the vitamin C release experiment. The vitamin C concentration gradually decreased after the first 12 hours and approached zero after 7 days.

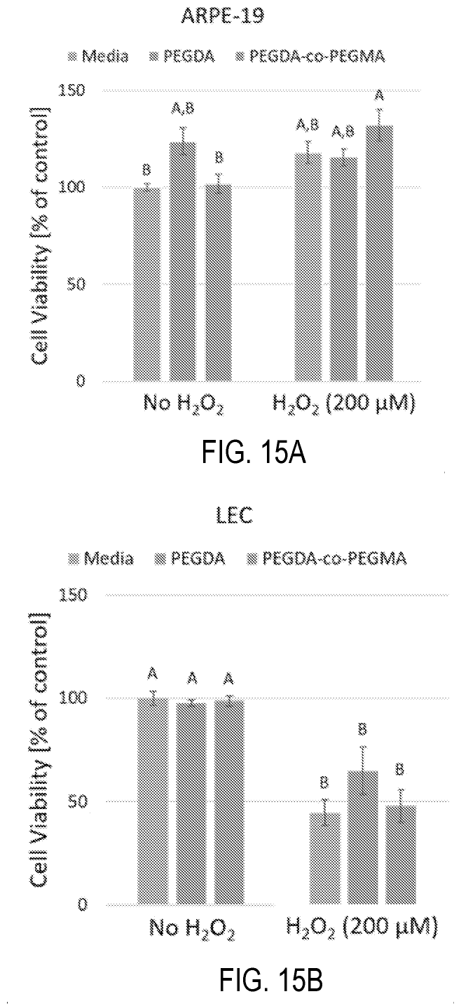

CellTiter-Glo luminescent cell viability assay showed that the hydrogels were not toxic to either ARPE-19 or LECs in vitro (FIGS. 15A-15B). The viability of cells cultured in media with hydrogels was not statistically different from the control with normal media. When compared to controls, hydrogen peroxide, used to introduce ROS, decreased the viability of LECs, less so for ARPE-19 cells. The viability of ARPE-19 cells treated with hydrogen peroxide was approximately the same or even higher compared to the non-treated groups. In contrast, the viability of LEC treated with hydrogen peroxide was statistically lower than that of the LEC without the hydrogen peroxide treatment, showing that, under these culture conditions, the lens cells are more sensitive to oxidative damage than ARPE-19 cells.

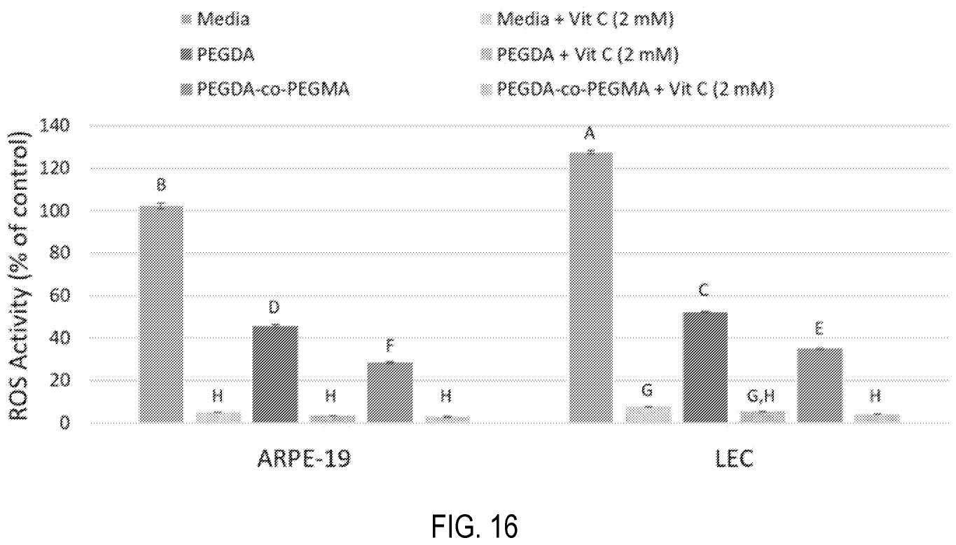

The DCF assay showed the protective effect of the hydrogels and vitamin C against ROS for ARPE-19 and LECs (FIG. 16). The ROS activity statistically decreased in the presence of either PEGDA or PEGDA-co-PEGMA hydrogels and further decreased with the addition of vitamin C, when compared to the control. Again, the hydrogen peroxide treatment did not affect the ROS activity of ARPE-19 cells. In contrast, compared to control, ROS activity of LEC increased with the addition of hydrogen peroxide. These results suggest that ARPE-19 cells were an appropriate control against more ROS-sensitive LECs.

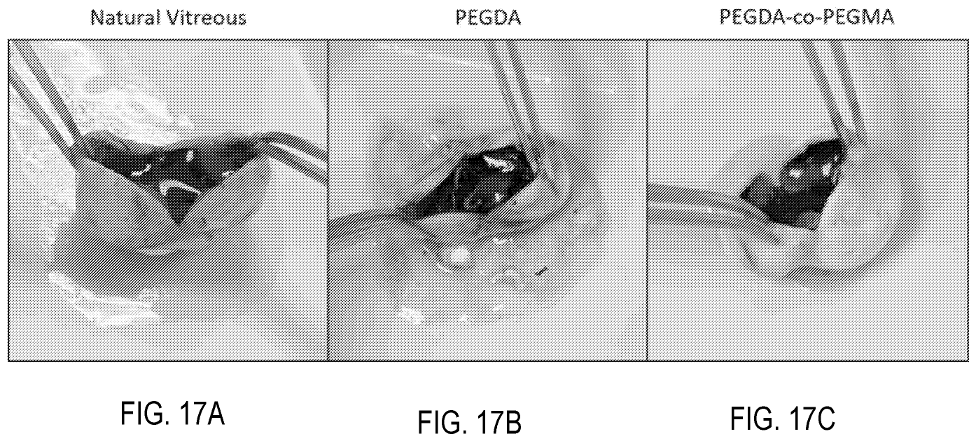

The hydrogels were successfully injected into the vitreal chamber of porcine eyes ex vivo (FIG. 17). The injected hydrogels were transparent and had similar consistency and appearance as the natural vitreous.

### Example 8. Physical and Chemical Methods to Improve Vitamin C Stability in Hydrogen Vitreous Substitutes

Due to the rapid degradation of Vitamin C in solution, typically in less than one week, both physical (encapsulating in multilayered particles) and chemical methods (mixing with glutathione) for stabilizing Vitamin C were examined in hydrogel vitreous substitutes.

Copolymers of poly(ethylene glycol) methacrylate (PEGMA) and poly(ethylene glycol) diacrylate (PEGDA) were prepared by free radical polymerization and loaded with Vitamin C (2 mM). To prepare physically-protected Vitamin C, chitosan (1 mg/mL) was crosslinked with sodium tripolyphosphate (1.75 mg/mL), loaded with Vitamin C (10% w/v), and coated with alternating layers of alginate (1 mg/mL) and chitosan. To chemically protect Vitamin C, glutathione solutions (1, 2, 4, or 10 mM) were instead added to chemically recycle Vitamin C. Either the particle solutions or the chemically-stabilized Vitamin C solutions were incubated in the hydrogels at 37° C. At predetermined times (0, 1, 2, 3, 4, 7, 8, 9, 11, and 14 days), the remaining Vitamin C was determined using a microplate reader at wavelength 265 compared to standard solutions with known concentrations with blank particles and glutathione solutions as the background readings.

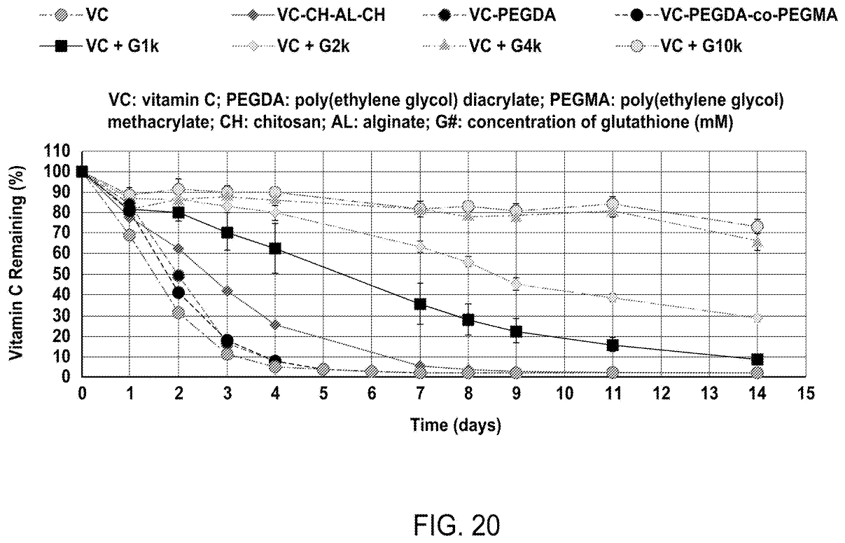

As shown in FIG. 20, the PEDGA and PEDGA-co-PEGMA hydrogels were injectable and appeared similar to the natural vitreous humor. Solutions containing only Vitamin C (with no hydrogel) degraded quickly to 0% by day 5. The hydrogels and particles provided some protection to the Vitamin C, leading to degradation after only 7 days. Glutathione as an additive provided the longest stabilization, with 70% of the Vitamin C remaining after 14 days when the glutathione concentration was greater than 4 mM. Blank hydrogels, particles, and glutathione solutions did not interfere with absorbance reading for Vitamin C.

Therefore, combining Vitamin C with glutathione significantly improved the stability of the Vitamin C for at least two weeks. Therefore, glutathione may prove to be an effective addition to Vitamin C loaded hydrogel vitreous substitutes to improve the stability of the included Vitamin C.

Materials and Methods: Ascorbic acid (VC), chitosan (CH, low molecular weight), alginate (AL), gelatin (GE), glutathione (GLU, St. Louis, Mo., USA), sodium tripolyphosphate (TPP, 85%), acetic acid and Dulbecco's phosphate-buffered saline (DPBS) were purchased from Sigma-Aldrich and used without further purification. Poly(ethylene glycol) methacrylate (PEGMA, average molecular weight (MW) 360), poly(ethylene glycol) diacrylate (PEGDA, average MW 575), N,N,N′,N′-Tetramethylethylenediamine (TEMED), ammonium persulfate (APS), and Dulbecco's phosphate-buffered saline (DPBS) were also purchased from Sigma-Aldrich (St. Louis, Mo., USA) and used for the preparation of hydrogel vitreous substitute. Dialysis tubing with molecular weight cut off (MWCO) of 6-8 kDa and 12-14 kDa, Dulbecco's Modified Eagle's/Nutrient Mixture F-12 Ham's Medium (DME/F-12), Dulbecco's Modified Eagle's Medium (DMEM), DMEM without phenol red, fetal calf serum (FCS), Penicillin-Streptomycin (Pen Strep), and hydrogen peroxide were purchased from Thermo Fisher Scientific (Waltham, Mass., USA) and used as received. RPE (ARPE-19 ATCC CRL-2302) were purchased from American Type Culture Collection (Manassas, Va., USA). Immortalized SRA 01/04 human LEC was originally provided by Dr. Venkat N. Reddy, University of Michigan and shared by Dr. Marlyn P. Langford, La. State University. The cell line was produced by transfection of human epithelial cells with plasmid vector DNA containing a large T antigen of SV40.33 (see Ibaraki, N. et al., Exp Eye Res 1998, 67, 577-585). CellTiter-Glo Luminescent Cell Viability Assay was purchased from Promega (Madison, Wis., USA). Dichlorofluorescein (2,7-Dichlorodihydrofluorescein diacetate, DCF) was purchased from Cayman Chemical (Ann Arbor, Mich., USA).

Preparation of Chitosan/Alginate/Gelatin Particles: Chitosan (Sigma-Aldrich, low molecular weight, 1 mg/mL) was dissolved in acetic acid solutions (1% w/w, 500 mL) for 60 min at 500 rpm. Sodium tripolyphosphate (1.75 mg/mL, 500 mL) was added dropwise into the chitosan solution to form nanoparticles for a 2-hour duration (see Liu, W. et al., LWT 2017, 75-608-615). The nanoparticles were collected via centrifugation at 4000 rpm for 15 min at 21° C. The particles were washed with deionized water and centrifuged once more. Ascorbic acid (10% w/w, 10 mL, pH 5.5) was added to the particles and let dissolve for 18 hours on an orbital shaker. Sodium alginate (FMC BioPolymer, Protanal PH, 1 mg/mL, 10 mL, pH 5.5) was added to the ascorbic acid and chitosan particle solution and sonicated for 30 min. Chitosan (Sigma-Aldrich, low molecular weight, 1 mg/mL in 1% w/w acetic acid solution, 10 mL) was added to the ascorbic acid-chitosan-alginate particles and sonicated for 30 min. In a different group, gelatin (bloom 175, 1 mg/mL 10 mL) was added to the ascorbic acid-chitosan-alginate particles and sonicated for 30 min. The particles were collected by centrifugation at 4000 rpm for 15 min at 21° C. and freeze dried.

Preparation of Hydrogels: The hydrogels were formed by free radical polymerization as previously published with modifications (see Tram, N. K. et al., Macromolecular Bioscience 2019, 1900305). Briefly, PEGMA, and PEGDA monomers were dissolved in deionized water and extensively purged with nitrogen gas to remove oxygen molecules that might terminate the reaction prematurely. APS aqueous solution (10% w/v) and TEMED were added as free radical initiator and accelerator at 1:200 and 1:800 v/v, respectively. The solutions were allowed to polymerize for 12 h. The hydrogels were purified against deionized water for 7 days in dialysis tubing (12-14 kDa MWCO) to remove unreacted monomers and low molecular weight polymer chains. Two optimized formulations were created, namely PEGDA (100% PEGDA, 2% wt polymer) and PEGDA-co-PEGMA (50% PEGDA:50% PEGMA, 3% wt polymer) hydrogels.

Vitamin C Release Study: The various solutions made were chitosan (CH), chitosan-alginate (CH-AL), chitosan-alginate-chitosan (CH-AL-CH), chitosan-alginate-gelatin (CH-AL-GE), and glutathione concentrations (GLU) at 0.1 uM, 1 uM, 10 uM, 100 uM, 1 mM, 2 mM, 4 mM, and 10 uM. One release study tested the combination of encapsulating particles and glutathione (1 mM) in the solutions CH-GLU, CH-AL-GLU, CH-AL-CH-GLU, CH-AL-GE-GLU as well as encapsulating both vitamin C and glutathione using the same layering methods. For each solution tested, a control group with no vitamin C and a test group with vitamin C (1% w/w) alone in DPBS were included. All groups tested had a target concentration of vitamin C at 2 mM. Other control groups included a VC-only solution (1% w/w in DPBS) and a glutathione-only solution (1% w/w). All solutions were kept at 37° C. throughout the release studies. The amount of viable vitamin C remaining in the solutions was measured using a Synergy HT multi-mode microplate reader (BioTek, Winooski, Vt.) at wavelength 265 nm and compared to the control groups to determine the amount of viable vitamin C left in the solutions. To measure the concentration of vitamin C, solutions with particles were centrifuged at 3220 rpm for 5 min and solution (500 uL) was removed and placed into a 96-well plate measured on days 0, 1, 2, 3, 4, 7, 8, 9, 10, 11, and 14. After two weeks, solutions with higher concentrations of vitamin C were measured every other day until vitamin C was undetectable. For the particle solutions, DPBS solutions (500 uL) was added back into the solution to maintain a constant volume.

Screening of Hydrogen Peroxide, Vitamin C, and Glutathione Concentrations Using a Cell Viability Assay: ARPE-19 and LEC were seeded in 96-well plates at 1×10⁴ cells per well in DMEM/F-12 and DMEM, respectively, supplemented with 10% FCS and 1% Pen Strep for 24 h at 37° C. in 5% CO₂ humidified atmosphere. The culture medium in each well was removed and various media with vitamin C (2000 μM, 1000 μM, 500 μM, 100 μM, and 0 μM) or glutathione (10000 μM, 4000 μM, 2000 μM, 1000 μM, 500 μM, and 0 μM) was added to each well (100 μL) and incubated for 24 hours. Hydrogen peroxide (600 μM, 400 μM, 200 μM, 100 μM, 50 μM, and 0 μM) and a special case of vitamin C (2000 μM) was added (100 μL) 30 minutes before performing the viability assays. CellTiter-Glo luminescent cell viability assay was conducted according to the manufacturer's protocol. Briefly, the well plates were equilibrated to room temperature for 30 minutes. CellTiter-Glo Reagent (100 μL) was added to each well, and the contents were mixed for 10 minutes using an orbital shaker. The well plates were incubated at room temperature for 10 minutes before the luminescent signal was measured using a Synergy HT multi-mode microplate reader (BioTek, Winooski, Vt.).

Antioxidant Activity of Vitamin C in Reducing Reactive Oxygen Species (ROS) Activity Using DCF Assay: The ROS activity induced by hydrogen peroxide (200 μM for 30 minutes) of ARPE-19 and LEC treated with vitamin C (0, 100, and 1000 μM for 30 minutes or 24 hours) was determined using dichlorofluorescein assay. Briefly, LEC and ARPE-19 cells were cultured as aforementioned. DCF (100 μL, 20 μm final concentration) was added to each well, and the contents were incubated at room temperature for 30 min (see Ou, Y. et al. Chem Biol Interact. 2009, 179, 103-109). The fluorescence signal was measured with excitation and emission wavelengths of 485 and 525 nm, respectively, using a Synergy HT multi-mode microplate reader (BioTek, Winooski, Vt.).

Statistical Analysis: Data were expressed as mean±standard error (SE). Statistical analyses were implemented with Minitab software (version 18.1; Minitab, Inc., State College, Pa.). One-way ANOVA, with post hoc pairwise comparison using Tukey's test, was used to analyze the cell viability and ROS activity of the LEC and ARPE-19 cells. The null hypotheses stated that there was no difference between the groups for each test. An alpha value of 0.05 was used for statistical significance.

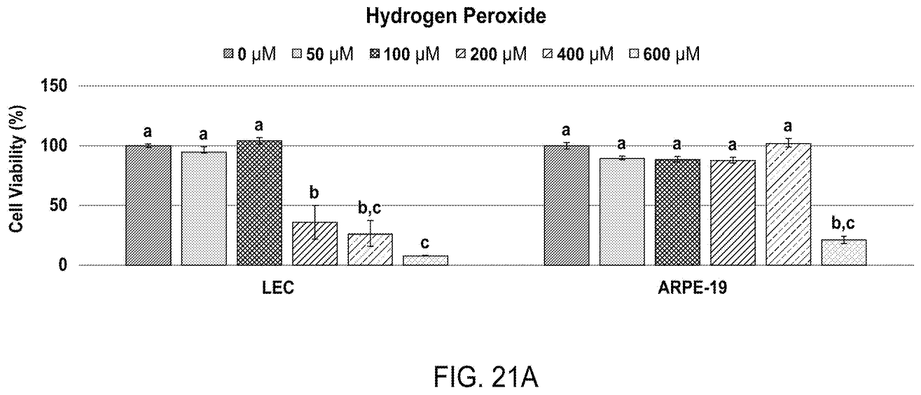

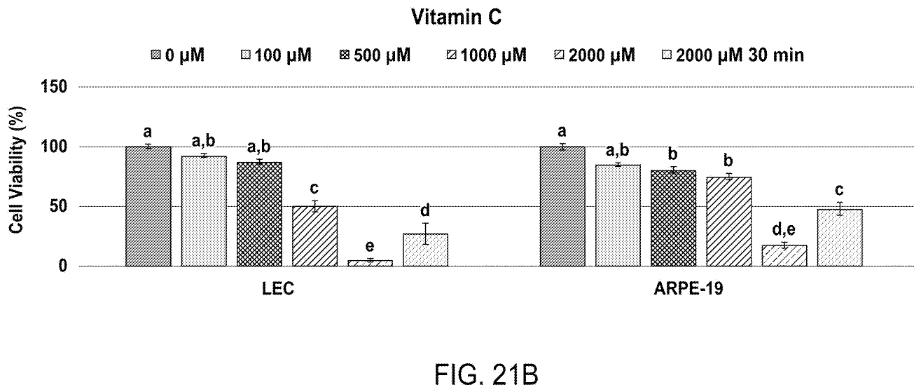

Results: Hydrogen peroxide did not affect cell viability at or below 100 μM and significantly decreased cell viability at 600 μM for both LEC and ARPE-19 (see FIG. 21A). Intermediate concentrations of H₂O₂ (200 μM and 400 μM) significantly decreased the cell viability of LEC but had no effect on ARPE-19. This result suggested that, under these culture conditions, the lens cells are more sensitive to oxidative damage than ARPE-19 cells, making ARPE-19 cells an appropriate control against more ROS-sensitive LECs. Vitamin C had an adverse effect on cell viability for both LEC and ARPE-19 (see FIG. 21B). While low concentration vitamin C (100 μM and 500 μM) could be considered nontoxic (above 70% cell viability), higher concentrations (1000 μM and 2000 μM) significantly decreased cell viability, even with reduced exposure time (2000 from 24 hours to 30 minutes).

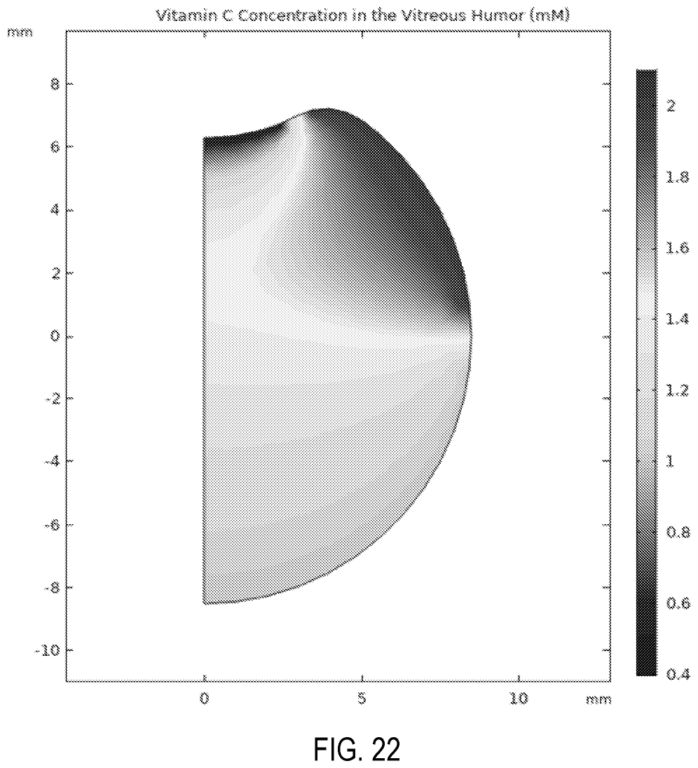

Vitamin C was toxic to retinal and lens epithelial cells at physiological vitreous concentrations (at or above 1000 Previous studies corroborated with the presented data and showed that 100 μM was the optimal concentration at preventing oxidative damage (see Goyal, M. M. et al. Indian J Clin Biochem. 2009, 24, 375-380; and Wei, W. et al. Scientific World Journal 2014, 750634). The results suggest the existence of a vitamin C gradient between the vitreous core and the vitreous cortex (in proximity with the cells), analogous to the previously established oxygen gradient in the vitreous humor (see Filas, B. A. et al. Invest Ophthalmol Vis Sci. 2013, 54, 6549-6559). This idea is illustrated in FIG. 22.

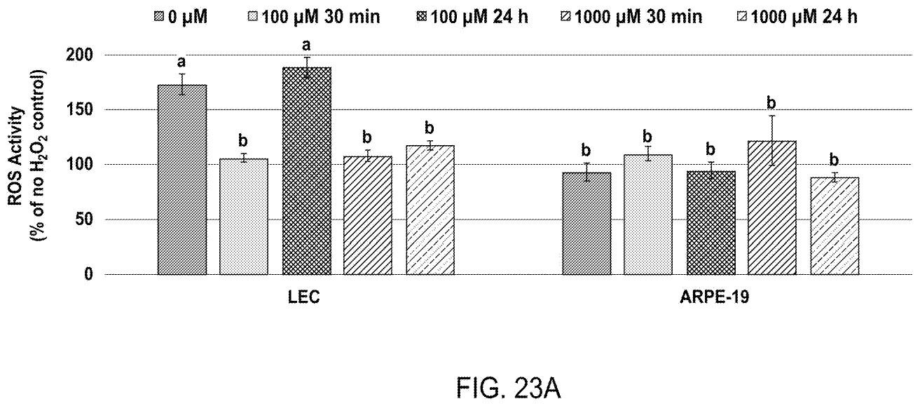

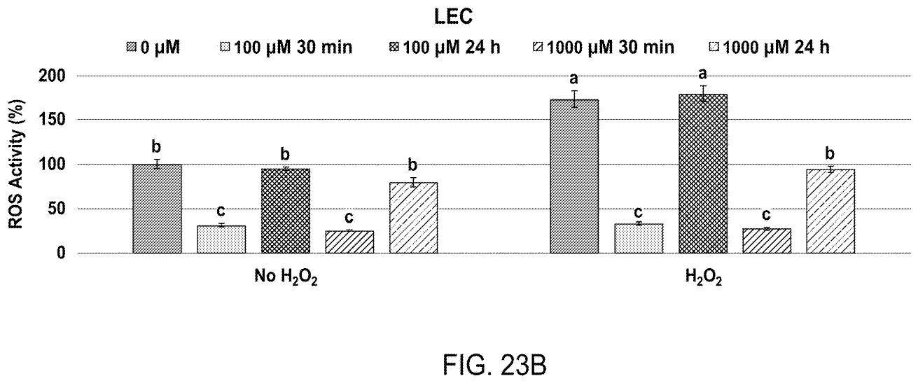

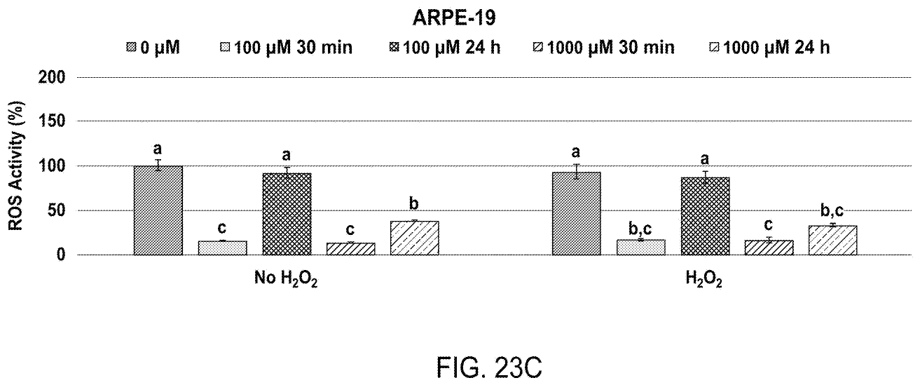

Vitamin C can reduce ROS activity of cells when used at high concentration (1000 μM) and/or when incubated simultaneously with hydrogen peroxide (see FIG. 23A). Low concentration of vitamin C (100 μM) incubated with cells for 24 hours was not effective at reducing ROS induced by hydrogen peroxide, thereby having the same ROS activity as the no vitamin C control. There was a 1.5 times increase in ROS activity in LECs treated with hydrogen peroxide (200 μM) (see FIG. 23B). LECs treated with H₂O₂ and high concentration of vitamin C (1000 μM) had the same ROS activity as the no H₂O₂ no vitamin C control. ARPE-19 cells, as previously determined, did not significantly respond to oxidative damage induced by hydrogen peroxide (see FIG. 23C). Treating cells with vitamin C at both high and low concentration for 30 minutes significantly reduced ROS activity to 15-30%. ROS activity returned to the same level as the control (no vitamin C) after 24 hours at low concentration of vitamin C (100 μM) for both cells when not treated with H₂O₂.

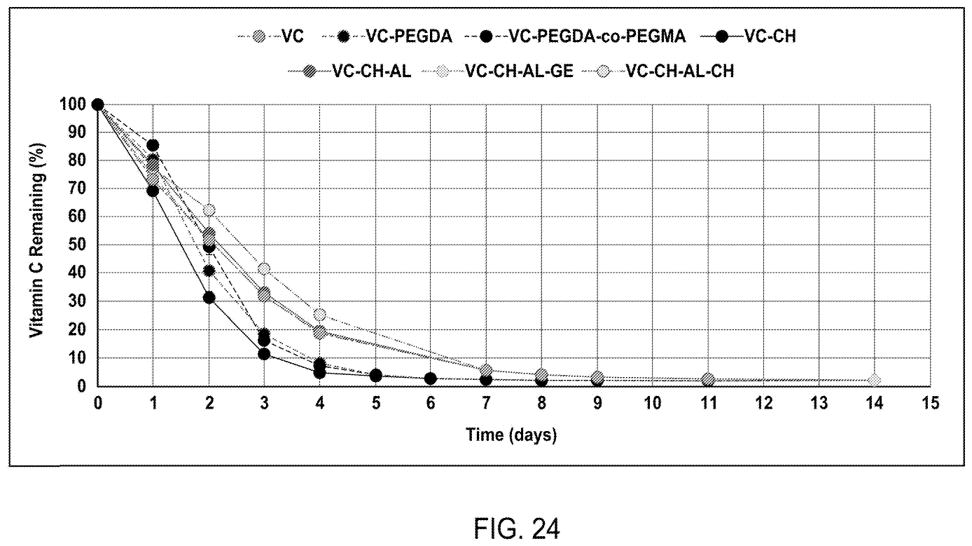

Vitamin C degrades rapidly to 10% after 3 days (see FIG. 24). Hydrogels improved the vitamin C remaining to 20% at day 3. Encapsulating vitamin C in chitosan, chitosan-alginate, and chitosan-alginate-gelatin particles increased the percent remaining to 30%, with chitosan-alginate-chitosan particles provided the best protection with 40% remaining after 3 days. All formulations approached 0% after 14 days.

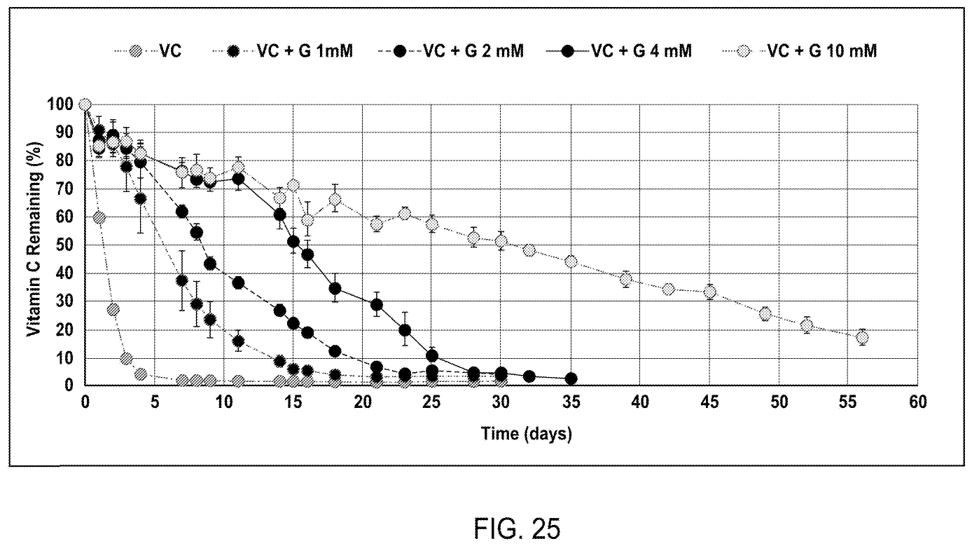

Mixing vitamin C with glutathione provided better protection to vitamin C than other physical methods (encapsulating in hydrogels or particles). The percent vitamin C remaining increased with the amount of glutathione used (see FIG. 25). More than half of the vitamin C remained past 14 days when combined with high concentrations of glutathione (4-10 mM).

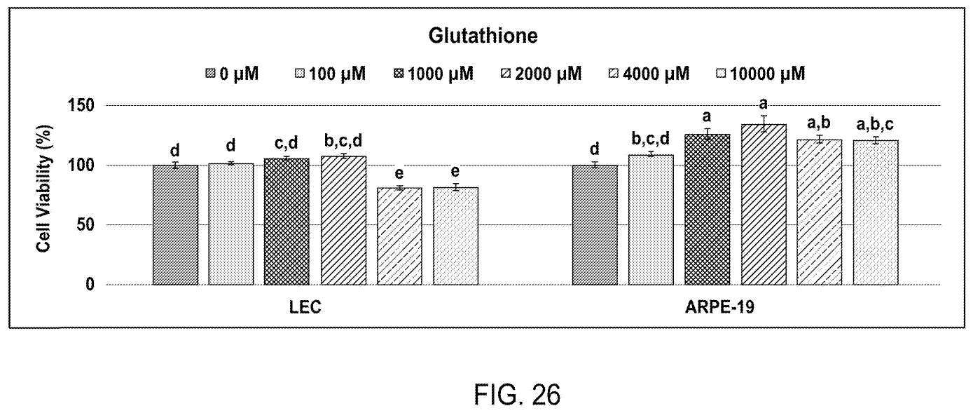

Glutathione was nontoxic to both cell types, even at high concentration (10000 μM), with cell viability staying above 70% for all tested conditions (see FIG. 26). LEC cell viability decreased at 4000 μM and 10000 but still stayed above 70%. ARPE-19 had increased cell viability with glutathione concentration above 100 μM.

It can be apparent to those skilled in the art that various modifications and variations can be made in the present disclosure without departing from the scope or spirit of the disclosure. Other embodiments of the disclosure can be apparent to those skilled in the art from consideration of the specification and practice of the disclosure disclosed herein. It is intended that the specification and examples be considered as exemplary only, with a true scope and spirit of the disclosure being indicated by the following claims.

1. A vitreous substitute comprising:
a gel; and
at least one antioxidant;
wherein the vitreous substitute is defined by having a loss tangent of less than 1 and a refractive index from about 1.33 to about 1.34.

2. The vitreous substitute of claim 1, having a loss tangent ranging from about 0.1 to about 0.5.

3. (canceled)

4. (canceled)

5. (canceled)

6. (canceled)

7. (canceled)

8. The vitreous substitute of claim 1, having a refractive index from about 1.331 to about 1.339 or from about 1.334 to about 1.337.

9. (canceled)

10. The vitreous substitute of claim 1, wherein the gel comprises a hydrogel, and wherein the hydrogel comprises a polymer composition.

11. (canceled)

12. (canceled)

13. (canceled)

14. (canceled)

15. The vitreous substitute of claim 10, wherein the polymer composition comprises one or more residues selected from poly(ethylene glycol)diacrylate (PEDGA), poly(ethylene glycol)methacrylate (PEGMA), 2-hydroxyethylmethacrylate (HEMA), or combinations thereof.

16. The vitreous substitute of claim 15, wherein the polymer composition comprises one or more PEGMA residues, and wherein each of the one or more PEGMA residues have a molecular weight from about 100 to about 500, from about 200 to about 400, from about 250 to about 400, or from about 280 to about 300.

17. (canceled)

18. (canceled)

19. (canceled)

20. (canceled)

21. The vitreous substitute of claim 15, wherein the polymer composition comprises one or more PEGDA residues, and wherein each of the one or more PEGDA residues have a molecular weight from about 100 to about 1000, from about 200 to about 1000, from about 300 to about 1000, from about 400 to about 1000, or from about 500 to about 900.

22. (canceled)

23. (canceled)

24. (canceled)

25. (canceled)

26. (canceled)

27. The vitreous substitute of claim 15, wherein the polymer composition comprises a PEGMA:PEGDA copolymer.

28. (canceled)

29. The vitreous substitute of claim 15, wherein the polymer composition comprises a PEGMA:PEGDA:HEMA copolymer.

30. (canceled)

31. (canceled)

32. The vitreous substitute of claim 1, further comprising a particle.

33. (canceled)

34. (canceled)

35. (canceled)

36. (canceled)

37. (canceled)

38. The vitreous substitute of claim 1, wherein the at least one antioxidant comprises ascorbic acid or a derivative thereof.

39. The vitreous substitute of claim 38, wherein ascorbic acid or a derivative thereof is present at a concentration from about 0.1 mM to about 5 mM or from about 0.1 mM to about 1 mM.

40. (canceled)

41. The vitreous substitute of claim 1, wherein the at least one antioxidant comprises a glutathione.

42. (canceled)

43. The vitreous substitute of claim 41, wherein the glutathione is present at a concentration from about 1 mM to about 100 mM or from about 4 mM to about 10 mM.

44. (canceled)

45. (canceled)

46. The vitreous substitute of claim 1, further comprising one or more additional therapeutic agents.

47. (canceled)

48. (canceled)

49. (canceled)

50. (canceled)

51. (canceled)

52. (canceled)

53. (canceled)

54. A method for treating an ophthalmological disorder in an eye of a subject in need thereof comprising injecting into the eye of the subject a therapeutically effective amount of the vitreous substitute of claim 1.

55. The method of claim 54, wherein the ophthalmological disorder comprises macular degeneration (MD), vitelliform degeneration of BEST, Stargardt disease, juvenile macular dystrophy, Behr's disease, Sorsby's dystrophy, Doyne honeycomb retinal dystrophy, a retinal tear, or proliferative retinopathy, or
wherein the ophthalmological disorder comprises one or more symptoms related to macular degeneration selected from: drusen surrounded by white-yellow spots; submacular discoid scar of tissues; choroidal neovascularization; detached pigment retinal epithelium (PED); atrophy of pigment retinal epithelium (RPE); anomalous expansion of choroidal blood vessels; blurred or disturbed vision area; central dead point pigment anomalies; mixed layer of thin granulations located on the inner side of Bruch's membrane; or thickening and lowered permeability of Bruch's membrane.

56. The method of claim 55, wherein the MD comprises atrophic (dry) MD, exudative (wet) MD, age-related macular retinopathy (ARM), choroidal neovascularization, detached pigment retinal epithelium (PED), or atrophy of pigment retinal epithelium (RPE).

57. (canceled)

58. (canceled)

59. (canceled)

60. (canceled)

61. The method of claim 54, wherein the subject has been diagnosed with or is at risk of developing a cataract.

62. (canceled)

63. The method of claim 54, wherein the vitreous substitute is administered following a vitrectomy.
